(19) United States
(12) Patent Application Publication
(10) Pub. No.: US 2017/0189409 A1
(43) Pub. Date: Jul. 6, 2017

(54) MEDICAL USE

(71) Applicant: Almirall, S.A., Barcelona (ES)

(72) Inventors: Nuria GODESSART MARINA, Barcelona (ES); Cristina BALAGUE PELAEZ, Barcelona (ES)

(21) Appl. No.: 15/313,722

(22) PCT Filed: May 21, 2015
(86) PCT No.: PCT/EP2015/061308

§ 371 Date: Nov. 23, 2016

(30) Foreign Application Priority Data
May 27, 2014 (EP) 14382192.4
Oct. 17, 2014 (EP) 14382400.1
Oct. 17, 2014 (EP) 14382401.9

(51) Int. Cl. A61K 31/53; C07D 487/04

(52) U.S. Cl. CPC A61K 31/53; C07D 487/04

(57) ABSTRACT

The present invention provides a compound, which is an inhibitor of phosphoinositide 3-kinase delta or a pharmaceutically acceptable salt and/or solvate thereof, for use in the treatment of an immunobullous skin disease mediated by autoantibodies by oral administration.

### FIELD OF THE INVENTION

The present invention is directed to new medical uses for phosphoinositide 3-Kinase delta (PI3K delta) inhibitors. Specifically, the present invention is directed to the use of such inhibitors in the treatment of immunobullous skin diseases mediated by autoantibodies, in particular pemphigus vulgaris, by oral administration.

### BACKGROUND OF THE INVENTION

Immunobullous skin diseases mediated by autoantibodies (also known as autoimmune blistering diseases or AIBDs) are a group of rare skin disorders characterized by IgG (or less often IgA) autoantibodies that attack adhesive proteins of the epidermis or the dermal-epidermal junction. These disorders present as blisters and erosions of the skin and/or mucous membranes. They can affect individuals of any age including children. In Germany, there are an estimated 2000 new cases of AIBDs per year, with an overall prevalence of about 12,000 cases. The incidence of the related diseases epidermolysis bullosa acquista (EBA) and the pemphigoid disorders is around 1 and 25 new cases per/million residents, respectively (Schmidt E, Zillikens D. Dermatol Clin 2011; 29:663-71; Joly P. J Inv Derm 2012; 132: 1998-04; Bertram F. J. Dtsch Derm Ges 2009; 7: 434-9.).

Immunobullous skin diseases mediated by autoantibodies are well known in the art and include intraepidermal immunobullous diseases, such as pemphigus vulgaris, pemphigus vegetans, pemphigus foliaceus, endemic pemphigus foliaceus, intercellular IgA dermatosis, paraneoplastic pemphigus; and subepidermal immunobullous diseases, such as bullous pemphigoid, mucous membrane pemphigoid, pemphigoid gestationis, linear IgA disease, epidermolysis bullosa acquisita, bullous systemic lupus erythematosus and dermatitis herpetiformis.

Pemphigus is a chronic immunobullous skin disease mediated by autoantibodies that causes painful blisters on skin and mucosae. The two main types of pemphigus are p. vulgaris (PV) and p. foliaceus and both are potentially lethal. PV is the most common form of pemphigus in the EU, accounting for 70-80% of all cases (Schmidt E, Zillikens D. Dermatol Clin 2011; 29:663-71; Joly P. J Inv Derm 2012; 132: 1998-04; Bertram F. J. Dtsch Derm Ges 2009; 7: 434-9.). Patients develop blisters that break almost immediately, leaving ulcerated sores. Both, cutaneous and mucosal lesions, are slow to heal, lead to major general discomfort, loss of body proteins, increase susceptibility to infection, and difficulties in eating and drinking (Kneisel A, Hertl M. J. Dtsch Derm Ges. 2011; 9(10):844-56).

Most pemphigus forms display serum IgG autoantibodies that target desmogleins (Dsg), which are components of desmosomes (adhesive complexes between keratinocytes) and induce loss of cell adhesion, eventually leading to blistering. Autoantibody-induced impairment of distinct Dsg isoforms causes either the mucosal form of PV (anti-Dsg3 IgG, oral mucosa lesions only), the mucocutaneous form of PV (anti-Dsg3 and anti-Dsg1 IgG, oral and skin lesions) or p. foliaceus (anti-Dsg1 IgG, skin lesions only). PV can be regarded as a prototypical B cell-mediated autoimmune disease where pathogenic IgG autoantibodies are the direct cause of the symptoms (Kneisel A, Hertl M. J. Dtsch Derm Ges. 2011; 9(11):927-47; Joly P. Clin Dermatol. 2011; 29(4):432-6.).

Pemphigus is estimated to affect anywhere from 0.7 to 5 people per 1,000,000 per year in the general population (NORD Rare Diseases Data Base, accessed October 2014). The incidence and proportion varies between territories (Meyer N, Misery L. Autoimmunity Reviews 2010; 9: A379-A382), but it is more prevalent in people of the Mediterranean area or Jewish ancestry. Men and women are equally affected. Although the onset usually occurs in middle-aged adults, the disease may also appear in young adults and children.

There is currently no cure for pemphigus vulgaris. The primary aim of current treatment is to decrease blister formation, prevent infections and promote healing of blisters and erosions. High dose corticosteroids (CS) are the standard of care (SOC) treatment for PV. CS act quickly and provide symptom relief, with long-term use being required to prevent relapses (maintenance of remission). However, 50% of patients remain poorly controlled after 1 year of treatment (Herbst A, Bystryn J C. J Am Acad Dermatol 2000; 42 (3), 422-427). In addition, long-term use of high dose CS increases the risks of side effects (morbidities and risk of mortality). To palliate this, adjuvant therapies are used as CS-sparing drugs to reduce CS side effects (azathioprine, mycophenolate mofetil, rituximab, methotrexate, IgG, cyclophosphamide, cyclosporine) but have not provided any additional efficacy over CS alone. Currently, there is a lack of alternative treatment to PV, with an improved efficacy/balance over current SOC.

The mortality rate of PV is about 5-15% (Schmidt E, Zillikens D. Dermatol Clin 2011; 29:663-71; Joly P. J Inv Derm 2012; 132: 1998-04; Bertram F. J. Dtsch Derm Ges 2009; 7: 434-9.). Mortality in patients with PV is 3 times higher than the general population, mainly due to the side effects of the current standard of care (SOC), high-dose CS including peptic ulcer disease and GI bleeds, and susceptibility to infection with sepsis. Morbidity and mortality are related to the extent of disease, the maximum dose of CS required to induce remission, and the presence of other diseases. Current morbidity of PV is largely iatrogenic, caused by side effects of the long-term high-dose CS and immunosuppressive adjuvants.

New and more effective therapies are therefore needed in the treatment of immunobullous skin diseases mediated by autoantibodies, in particular pemphigus vulgaris.

It has now been surprisingly found that phosphoinositide 3-Kinase delta (PI3K delta) inhibitors are effective in the treatment of immunobullous skin diseases mediated by autoantibodies, in particular pemphigus vulgaris. This opens up a new treatment pathway for such diseases which may avoid the problems associated with existing steroid and immunosuppressant/immunomodulatory therapies. It is a finding of the invention that PI3K delta inhibitors are particularly effective when administered orally, as compared with administration by other means, eg topical administration.

Treament of immunobullous skin diseases mediated by autoantibodies with phosphoinositide 3-Kinase delta (PI3K delta) inhibitors advantageously targets B-lymphocyte functions and reduces pathogenic IgG antibody titers against autoantigens associated with such diseases. In particular, treatment with phosphoinositide 3-Kinase delta (PI3K delta) inhibitors advantageously reduces the production of antibodies to Dsg3, which are associated with immunobullous skin diseases. Thus, treatment with such agents targets the underlying etiology of the disease (production of unwanted specific antibodies), rather than simply alleviating the symptoms or suppressing the immune system generally.

Phosphoinositide 3-Kinases (PI3Ks) are among enzymes involved in early intracellular signalling cascades involving the regulation of second messengers when cells are activated by extracellular stimuli. This eventually produces a response of the cell to the stimuli. PI3Ks phosphorylate the 3-hydroxyl group of the inositol ring of phosphatidylinositol (Ptdlns), Ptdlns-4-phosphate (Ptdlns4P), and Ptdlns-4,5-bisphosphate (Ptdlns(4,5)P2). The resulting 3-phosphoinositides mediate correct localization and subsequent activation of a number of downstream effector proteins that bind to the lipids via specific lipid binding sequences such as the pleckstrin homology (PH) domain (Vanhaesebroeck B, 2010, Nat Rev Mol Cell Biol 5:11381-6).

The PI3K family is divided into 3 different classes (PI3K class I, class II, and class III), depending on substrate preference and structural features.

The best characterized is the PI3K class I with the preferential substrate Ptdlns-(4,5)P2. It englobes 4 different isoforms which originally were further subdivided into class IA (p110a, p110b, p110d), binding to a p85 type of regulatory subunit, and class IB (p110g) which is regulated by p101 and p87 subunits. Whereas p110a (PI3Kα or PI3Kα) and p110b (PI3Kb or PI3Kβ) isoforms are expressed ubiquitously, p110g (PI3Kg or PI3Kγ) and especially p110d (PI3Kd or PI3Kδ) have a more restricted expression pattern and seem to play a major role in leukocytes (Kok K, Trends Biochem Science 34:115-127, 2009).

Several PI3K inhibitors are currently in clinical trials for the treatment or prevention of diseases or disorders known or suspected to be linked to the PI3K pathway. Examples include alpelisib (previously known as BYL-719), buparlisib (previously known as BKM 120 or NVP-BKM120), duvelisib (previously known as IPI-145 or INK-1197), idelalisib (previously known as GS-1101 or CAL-101), rigosertib sodium (previously known as ON-1910Na), and 6-(2-((4-amino-3-(3-hydroxyphenyl)-1H-pyrazolo[3,4-d]pyrimidin-1-yl)methyl)-3-(2-chlorobenzyl)-4-oxo-3,4-dihydroquinazolin-5-yl)-N, N-bis(2-methoxyethyl)hex-5-ynamide (also known as RV-1729).

### SUMMARY OF THE INVENTION

The present invention therefore provides a compound, which is an inhibitor of phosphoinositide 3-kinase delta or a pharmaceutically acceptable salt and/or solvate thereof, for use in the treatment of an immunobullous skin disease mediated by autoantibodies by oral administration.

The invention also provides a pharmaceutical composition for use in the treatment of an immunobullous skin disease mediated by autoantibodies as defined herein by oral administration, which composition comprises a compound as defined herein and a pharmaceutically acceptable carrier.

The present invention also provides use of a compound or composition as defined herein, for the manufacture of a medicament for the treatment of an immunobullous skin disease mediated by autoantibodies as defined herein by oral administration.

The invention also provides a method of treating an immunobullous skin disease mediated by autoantibodies as defined herein, which method comprises administering orally to a patient in need thereof an effective amount of a compound or composition as defined herein.

### BRIEF DESCRIPTION OF THE FIGURES

FIG. 1 shows the effect of a representative compound of the invention, LAS191954, and prednisolone on the kinetics of antibody production to Dsg3.

FIG. 2 shows the effect of a representative compound of the invention, LAS191954, and prednisolone on the kinetics of antibody production to dsDNA.

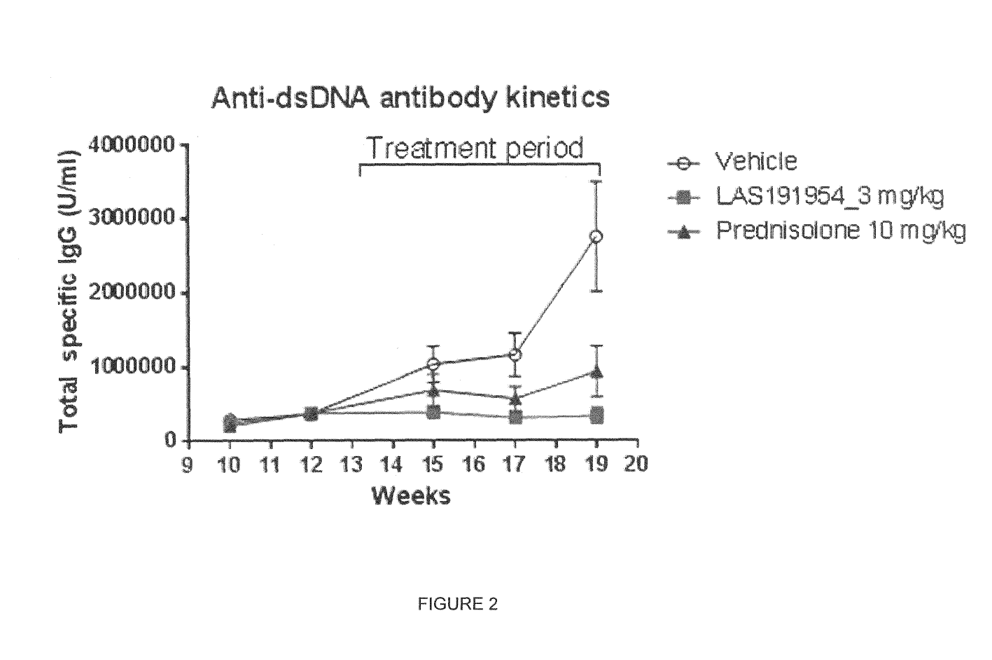

FIG. 3 shows the relative change of Anti-Dsg3 (Left) and Anti-dsDNA (Right) antibody levels in a spontaneous autoimmune disease model.

FIG. 4 shows the effect of a representative compound of the invention, LAS191954, on clinical disease in established experimental EBA as determined by the percentage of body surface area affected by skin lesions in relation to the score at inclusion to treatment.

FIG. 5 shows the effect of a representative compound of the invention, LAS191954, on clinical disease in established experimental EBA as determined by the overall disease activity, expressed as AUC derived from graphs in FIG. 4.

FIG. 6 shows the effect of a representative compound of the invention, LAS191954, on clinical disease in established experimental EBA as determined by representative clinical manifestations.

FIG. 7 shows the effect of a representative compound of the invention, LAS191954, on body weight gain in established experimental EBA.

### DETAILED DESCRIPTION OF THE INVENTION

The term “therapeutically effective amount” refers to an amount sufficient to effect treatment when administered to a patient in need of treatment.

The term “treatment” as used herein refers to the treatment of a disease or medical condition in a human or animal patient which includes:

(a) preventing the disease or medical condition from occurring, i.e., prophylactic treatment of a patient;
(b) ameliorating the disease or medical condition, i.e., causing regression of the disease or medical condition in a patient;
(c) suppressing the disease or medical condition, i.e., slowing the development of the disease or medical condition in a patient; and/or
(d) alleviating the symptoms of the disease or medical condition in a patient.

The term “pharmaceutically acceptable salt” refers to a salt prepared from a base or acid which is acceptable for administration to a patient, such as a human or animal e.g. a mammal. Such salts can be derived from pharmaceutically-acceptable inorganic or organic bases and from pharmaceutically-acceptable inorganic or organic acids.

Pharmaceutically acceptable acids include both inorganic acids, for example hydrochloric, sulphuric, phosphoric, diphosphoric, hydrobromic, hydroiodic and nitric acid; and organic acids, for example citric, fumaric, gluconic, glutamic, lactic, maleic, malic, mandelic, mucic, ascorbic, oxalic, pantothenic, succinic, tartaric, benzoic, acetic, methanesulphonic acid, ethanesulphonic, benzenesulphonic, naphthalene-2-sulfonic acid, p-toluenesulphonic acid, xinafoic (1-hydroxy-2-naphthoic acid), napadisilic (1,5-naphthalenedisulfonic acid) and the like. Particularly preferred are salts derived from methanesulphonic acid, naphthalene-2-sulfonic acid, and p-toluenesulphonic acid.

Salts derived from pharmaceutically-acceptable inorganic bases include aluminum, ammonium, calcium, copper, ferric, ferrous, lithium, magnesium, manganic, manganous, potassium, sodium, zinc and the like.

Salts derived from pharmaceutically-acceptable organic bases include salts of primary, secondary and tertiary amines, including alkyl amines, arylalkyl amines, heterocyclyl amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

The term “solvate” refers to a complex or aggregate formed by one or more molecules of a solute, i.e. a phosphoinositide 3-kinase delta inhibitor or a pharmaceutically acceptable salt thereof, and one or more molecules of a solvent. Such solvates are typically crystalline solids having a substantially fixed molar ratio of solute and solvent. Representative solvents include by way of example, water, acetone, dichloromethane, 2-propanol, ethanol, methanol, dimethylsulfoxide (DMSO), ethyl acetate, acetic acid, ethanolamine, or mixtures thereof. When the solvent is water, the solvate formed is a hydrate. It is specifically contemplated that one solvent molecule can be associated with one molecule of a phosphoinositide 3-kinase delta inhibitor or a pharmaceutically acceptable salt thereof, such as a hydrate. Furthermore, it is specifically contemplated that more than one solvent molecule may be associated with one molecule of a phosphoinositide 3-kinase delta inhibitor or a pharmaceutically acceptable salt thereof, such as a dihydrate. Additionally, it is specifically contemplated that less than one solvent molecule may be associated with a phosphoinositide 3-kinase delta inhibitor or a pharmaceutically acceptable salt thereof, such as a hemihydrate. Furthermore, solvates are contemplated as solvates of a phosphoinositide 3-kinase delta inhibitor or a pharmaceutically acceptable salt thereof that retain the biological effectiveness of the non-solvate form of the compounds.

The term “pharmaceutically (or physiologically) acceptable carrier (or diluent)” refers to a carrier or diluent that does not cause significant irritation to an organism and does not abrogate the biological activity and properties of the administered compound.

As used herein, the term “an inhibitor of phosphoinositide 3-kinase delta” refers to a compound that demonstrates activity against expression of phosphoinositide 3-kinase delta in a suitably chosen assay method, for instance an assay based on M-CSF-induced AKT phosphorylation, a downstream effector of PI3Kδ, in THP-1 cells.

Typically, “an inhibitor of phosphoinositide 3-kinase delta” refers to a compound that possesses an IC₅₀ value for the inhibition of PI3Kδ of less than 10 μm, preferably less than 1 μm, even more preferably less than 0.2 μm, most preferably less than 0.05 μm, for instance in the assay method referred to above.

Typically, the term “an inhibitor of phosphoinositide 3-kinase delta” refers to a compound that inhibits the activity of the PI3K delta isozyme more effectively than other isozymes of the PI3K family (alpha, beta, and gamma). For instance, the PI3-kinase delta selective inhibitor may refer to a compound that exhibits a 50 percent inhibitory concentration (IC₅₀) with respect to the delta type PI3-kinase that is at least 10-fold, preferably at least 20-fold, more preferably at least 50-fold, most preferably at least 100-fold or lower than the inhibitor's IC50 with respect to the rest of the other type PI3-kinases (i.e., alpha, beta, and gamma). Typically, this selectivity is determined using an assay method as defined above.

Typically, the inhibitor of phosphoinositide 3-kinase delta is a compound as defined in WO-A-2012/146666, the entirety of which is incorporated herein by reference.

Thus, typically, the inhibitor of phosphoinositide 3-kinase delta is of formula (I)

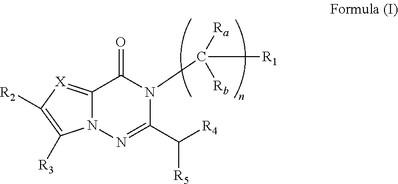

wherein X, Rₐ, R_{b}, n, R₁, R₂, R₃, R₄ and R₅ are as defined in WO-A-2012/146666.

Preferably, in the compound of formula (I):
X represents a nitrogen atom or a —CR₆ group;
Rₐ and R_{b} each independently represent a hydrogen atom or a methyl group;
R₁ represents a hydrogen atom, a halogen atom, a C₁-C₃ haloalkyl group, a methyl group, a C₃-C₇ cycloalkyl group, a phenyl group, a pyridinyl group, a pyrazolyl group, an isoxazolyl group, a piperidinyl group or a tetrahydropyranyl group; wherein the cycloalkyl, phenyl, pyridinyl, pyrazolyl, isoxazolyl, piperidinyl or tetrahydropyranyl groups are unsubstituted or substituted by one or more substituents selected from a halogen atom, a hydroxyl group, a C₁-C₃ haloalkyl group, a linear or branched C₁-C₃ alkyl group, a —(CH₂)-(phenyl)-O—(C₁-C₃ alkyl group), a —NR₇R₈ group or a —OR₈ group; wherein R₇ and R₈ each independently represent a hydrogen atom or a linear or branched C₁-C₃ alkyl group;
R₂ and R₃ each independently represent a hydrogen atom, a halogen atom, a cyano group, a C₁-C₃ haloalkyl group or a linear or branched C₁-C₃ alkyl group;
R₄ represents a hydrogen atom, a C₁-C₃ haloalkyl group, a C₁-C₃ hydroxyalkyl group or a linear or branched C₁-C₃ alkyl group;
R₆ represents a hydrogen atom, a halogen atom, a C₁-C₃ haloalkyl group, a linear or branched C₁-C₃ hydroxyalkyl group, a linear or branched C₁-C₃ alkyl group or a cyclopropyl group;
R₆ represents a hydrogen atom; a halogen atom; a hydroxyl group; a cyano group; a C₁-C₄ alkoxy group; a C₁-C₄ haloalkyl group; a linear or branched C₁-C₄ hydroxyalkyl group; a C₃-C₇ cycloalkyl group; a linear or branched C₁-C₃ alkyl group;
a —(CH₂)₀₋₃NR′R″ group; a —(CH₂)1-3O(C₁-C₃ alkyl group); a —(CH₂)₀₋₃OC(O)—(C₁-C₃ alkyl group); a —(CH₂)₀₋₃C(O)O—(C₁-C₃ alkyl group); a —C(O)—NR′R″ group; a —(CH₂)₀₋₃C(O)OH group; a —(CH₂)₀₋₃-(imidazolyl group); a —(CH₂)₀₋₃-(oxazolyl group); a —(CH₂)₀₋₃-(oxadiazolyl group); a —(CH₂)₀₋₃-(pyrazolyl group) or a —(CH₂)₀₋₃-(morpholinyl group); wherein R′ and R″ each independently represent a hydrogen atom, a hydroxyl group, or a linear or branched C₁-C₃ alkyl group; and wherein the imidazolyl, oxazolyl, oxadiazolyl, pyrazolyl and morpholinyl groups are unsubstituted or substituted by one or more substituents selected from a halogen atom, a linear or branched C₁-C₃ alkyl group or a C₁-C₃ haloalkyl group.
R₅ represents a group selected from:
i) a group of formula (IIa), which group is a purinyl group unsubstituted or substituted by a —NR′R″ group;
ii) a group of formula (IIb), which group is selected from a —NR′-pyridinyl group, a —S-pyridinyl group, a —NR′-pyrimidinyl group, a —S-pyrimidinyl group or a —NR′-triazinyl group; wherein the pyridinyl, pyrimidinyl and triazinyl groups are unsubstituted or substituted by one, two or three substituents selected from a halogen atom, a C₁-C₃ haloalkyl group, a —(CH₂)₀₋₃CN group, a —C(O)—(CH₂)₀₋₃—NR′R″, a —(CH₂)₀₋₃NR′R″ group; and
iii) a group of formula (IIc), which group is selected from a —NR′-purinyl group, a —S— purinyl group, a —NR′-7H-pyrrolo[2,3-d]pyrimidinyl group, a —NR′-1H-pyrazolo[3,4-d]pyrimidinyl group or a —NR′-pyrazolo[1,5-a]pyrimidinyl group; wherein the purinyl, 7H-pyrrolo[2,3-d]pyrimidinyl, 1H-pyrazolo[3,4-d]pyrimidinyl pyrazolo[1,5-a]pyrimidinyl and groups are unsubstituted or substituted by a halogen atom or a —(CH₂)₀₋₃NR′R″ group; or
R₄ and R₅ together with the carbon atom to which they are attached form a pyrrolidinyl-purinyl group or a pyrrolidinyl-pyrimidinyl; wherein the pyrrolidinyl group is unsubstituted or substituted by one or more substituents selected from a halogen atom or a hydroxyl group; and wherein the purinyl group is unsubstituted or substituted by
a —(CH₂)₀₋₃NR′R″ group; and wherein the pyrimidinyl group is unsubstituted or substituted by one, two or three substituents selected from a —(CH₂)₀₋₃CN group or a —(CH₂)₀₋₃NR′R″ group; and
R′ and R″ each independently represent a hydrogen atom, a C₁-C₃ alkoxy group or a linear or branched C₁-C₃ alkyl group.

Alternatively in the compound of formula (I):
X represents a nitrogen atom or a —CR₆ group;
Rₐ and R_{b} each independently represent a hydrogen atom or a methyl group;
R₁ represents a methyl group, a C₃-C₇ cycloalkyl group, a phenyl group, a pyridinyl group, a piperidinyl group or a tetrahydropyranyl group;
wherein the cycloalkyl, phenyl, pyridinyl, piperidinyl or tetrahydropyranyl groups are unsubstituted or substituted by one or more substituents selected from a halogen atom, a linear or branched C₁-C₃ alkyl group, a —NR₇R₈ group or a —OR₈ group; wherein R₇ and R₈ each independently represent a hydrogen atom or a linear or branched C₁-C₃ alkyl group;
R₂ and R₃ each independently represent a hydrogen atom or a linear or branched C₁-C₃ alkyl group;
R₄ represents a hydrogen atom, a C₁-C₃ haloalkyl group, or a linear or branched C₁-C₃ alkyl group;
R₆ represents a hydrogen atom, a halogen atom, a C₁-C₃ haloalkyl group, a linear or branched C₁-C₃ alkyl group or a cyclopropyl group; R₅ represents a group selected from:
i) a group of formula (IIa), which group is a purinyl group unsubstituted or substituted by a —NR′R″ group;
ii) a group of formula (IIb), which group is selected from a —NH-pyridinyl group, a —S-pyridinyl group, a —NH-pyrimidinyl group or a —S-pyrimidinyl group; wherein the pyridinyl or pyrimidinyl groups are unsubstituted or substituted by one, two or three substituents selected from a —(CH₂)₀₋₃CN group, a —C(O)—(CH₂)₀₋₃—NR′R″ or a —(CH₂)₀₋₃NR′R″ group; and
iii) a group of formula (IIc), which group is selected from a —NH-purinyl group or a —S-purinyl group; wherein the purinyl group is unsubstituted or substituted by a —(CH₂)₀₋₃NR′R″ group; or
R₄ and R₅ together with the carbon atom to which they are attached form a pyrrolidinyl-purinyl group, wherein the purinyl group is unsubstituted or substituted by a —(CH₂)₀₋₃NR′R″ group; and
R′ and R″ each independently represent a hydrogen atom, a C₁-C₃ alkoxy group or a linear or branched C₁-C₃ alkyl group.

More preferably, the compound of formula (I) is one of
2-((6-Amino-9H-purin-9-yl)methyl)-5-chloro-3-o-tolylpyrrolo[1,2-f][1,2,4]triazin-4(3H)-one;
2-((6-Aminopyrimidin-4-ylamino)methyl)-5-chloro-3-o-tolylpyrrolo[1,2-f][1,2,4]triazin-4(3H)-one;
2-((6-Amino-9H-purin-9-yl)methyl)-5-cyclopropyl-3-o-tolylpyrrolo[1,2-f][1,2,4]triazin-4(3H)-one;
2-((6-amino-9H-purin-9-yl)methyl)-3-o-tolylpyrrolo[1,2-f][1,2,4]triazin-4(3H)-one;
2-((6-aminopyrimidin-4-ylamino)methyl)-3-o-tolylpyrrolo[1,2-f][1,2,4]triazin-4(3H)-one;
4-((4-Oxo-3-o-tolyl-3,4-dihydropyrrolo[1,2-f][1,2,4]triazin-2-yl)methylamino)picolinamide;
2-((2-aminopyridin-4-ylamino)methyl)-3-o-tolylpyrrolo[1,2-f][1,2,4]triazin-4(3H)-one;
2-((9H-purin-6-ylamino)methyl)-3-o-tolylpyrrolo[1,2-f][1,2,4]triazin-4(3H)-one;
2-((6-Amino-9H-purin-9-yl)methyl)-3-cyclohexylpyrrolo[1,2-f][1,2,4]triazin-4(3H)-one;
2-((6-amino-9H-purin-9-yl)methyl)-5-methyl-3-o-tolylpyrrolo[1,2-f][1,2,4]triazin-4(3H)-one;
2-((9H-purin-6-ylthio)methyl)-5-methyl-3-o-tolylpyrrolo[1,2-f][1,2,4]triazin-4(3H)-one;
2-((6-amino-9H-purin-9-yl)methyl)-6-methyl-3-o-tolylpyrrolo[1,2-f][1,2,4]triazin-4(3H)-one;
2-((9H-purin-6-ylthio)methyl)-6-methyl-3-o-tolylpyrrolo[1,2-f][1,2,4]triazin-4(3H)-one;
2-(1-(6-amino-9H-purin-9-yl)ethyl)-3-phenylpyrrolo[1,2-f][1,2,4]triazin-4(3H)-one;
(S)-2-(1-(9H-purin-6-ylamino)propyl)-3-phenylpyrrolo[1,2-f][1,2,4]triazin-4(3H)-one;
(S)-2-(1-(6-aminopyrimidin-4-ylamino)propyl)-3-phenylpyrrolo[1,2-f][1,2,4]triazin-4(3H)-one;
(S)-2-(1-(2-amino-9H-purin-6-ylamino)propyl)-3-phenylpyrrolo[1,2-f][1,2,4]triazin-4(3H)-one;
(S)-4-amino-6-(1-(4-oxo-3-phenyl-3,4-dihydropyrrolo[1,2-f][1,2,4]triazin-2-yl)propylamino)pyrimidine-5-carbonitrile;
(R)-2-(1-(9H-purin-6-ylamino)propyl)-3-phenylpyrrolo[1,2-f][1,2,4]triazin-4(3H)-one;
(S)-2-(1-(9H-purin-6-ylamino)ethyl)-3-phenylpyrrolo[1,2-f][1,2,4]triazin-4(3H)-one;
(S)-2-(1-(2-amino-9H-purin-6-ylamino)ethyl)-3-phenylpyrrolo[1,2-f][1,2,4]triazin-4(3H)-one;
(S)-2-(1-(6-aminopyrimidin-4-ylamino)ethyl)-3-phenylpyrrolo[1,2-f][1,2,4]triazin-4(3H)-one;
(S)-4-amino-6-(1-(4-oxo-3-phenyl-3,4-dihydropyrrolo[1,2-f][1,2,4]triazin-2-yl)ethylamino)pyrimidine-5-carbonitrile;
2-(1-(6-amino-9H-purin-9-yl)ethyl)-5-methyl-3-phenylpyrrolo[1,2-f][1,2,4]triazin-4(3H)-one;
2-((6-Amino-9H-purin-9-yl)methyl)-3-o-tolyl-5-(trifluoromethyl)pyrrolo[1,2-f][1,2,4]triazin-4(3H)-one;
2-((6-Amino-9H-purin-9-yl)methyl)-5-chloro-3-phenylpyrrolo[1,2-f][1,2,4]triazin-4(3H)-one;
2-((6-Amino-9H-purin-9-yl)methyl)-5-chloro-3-(3-methoxyphenyl)pyrrolo[1,2-f][1,2,4]triazin-4(3H)-one;
2-((6-Amino-9H-purin-9-yl)methyl)-5-chloro-3-(2,4-difluorophenyl)pyrrolo-[1,2-f][1,2,4]triazin-4(3H)-one;
2-((6-Amino-9H-purin-9-yl)methyl)-3-benzyl-5-chloropyrrolo[1,2-f][1,2,4]-triazin-4(3H)-one;
2-((6-amino-9H-purin-9-yl)methyl)-3-phenylimidazo[1,2-f][1,2,4]triazin-4(3H)-one;
2-((6-amino-9H-purin-9-yl)methyl)-3-o-tolylimidazo[1,2-f][1,2,4]triazin-4(3H)-one;
2-((6-Amino-9H-purin-9-yl)methyl)-5-chloro-3-(pyridin-4-yl)pyrrolo[1,2-f][1,2,4]triazin-4(3H)-one;
2-((6-Amino-9H-purin-9-yl)methyl)-5-chloro-3-(tetrahydro-2H-pyran-4-yl)pyrrolo-[1,2-f][1,2,4]triazin-4(3H)-one;
2-((6-Amino-9H-purin-9-yl)methyl)-5-chloro-3-(1-methylpiperidin-4-yl)pyrrolo[1,2-f][1,2,4]triazin-4(3H)-one;
(S)-2-(1-(9H-Purin-6-ylamino)ethyl)-3-(3-fluorophenyl)pyrrolo[1,2-f][1,2,4]triazin-4(3H)-one;
(S)-4-Amino-6-(1-(3-(3-fluorophenyl)-4-oxo-3,4-dihydropyrrolo[1,2-f][1,2,4]triazin-2-yl)ethylamino)pyrimidine-5-carbonitrile;
(S)-2-(1-(9H-Purin-6-ylamino)ethyl)-3-(3,5-difluorophenyl)pyrrolo[1,2-f][1,2,4]triazin-4(3H)-one;
(S)-4-Amino-6-(1-(3-(3,5-difluorophenyl)-4-oxo-3,4-dihydropyrrolo[1,2-f][1,2,4]triazin-2-yl)ethylamino)pyrimidine-5-carbonitrile;
2-((6-Amino-9H-purin-9-yl)methyl)-5-chloro-3-methylpyrrolo[1,2-f][1,2,4]triazin-4(3H)-one;
2-((6-Amino-9H-purin-9-yl)methyl)-3-((1r,4r)-4-aminocyclohexyl)-5-chloropyrrolo[1,2-f][1,2,4]triazin-4(3H)-one;
(R)-2-((6-Amino-9H-purin-9-yl)methyl)-5-chloro-3-(1-phenylethyl)pyrrolo[1,2-f][1,2,4]triazin-4(3H)-one;
(S)-2-((6-Amino-9H-purin-9-yl)methyl)-5-chloro-3-(1-phenylethyl)pyrrolo[1,2-f][1,2,4]triazin-4(3H)-one;
(S)-4-amino-6-(1-(4-oxo-3-(pyridin-2-yl)-3,4-dihydropyrrolo[1,2-f][1,2,4]triazin-2-yl)ethylamino)pyrimidine-5-carbonitrile;
(S)-2-(1-(9H-purin-6-yl)pyrrolidin-2-yl)-3-phenylpyrrolo[1,2-f][1,2,4]triazin-4(3H)-one;
(S)-4-amino-6-(1-(4-oxo-3-phenyl-5-(trifluoromethyl)-3,4-dihydropyrrolo[1,2-f][1,2,4]triazin-2-yl)ethylamino)pyrimidine-5-carbonitrile;
(S)-2-(1-(9H-purin-6-ylamino)ethyl)-3-phenyl-5-(trifluoromethyl)pyrrolo[1,2-f][1,2,4]triazin-4(3H)-one;
(S)-4-amino-6-(1-(5-(difluoromethyl)-4-oxo-3-phenyl-3,4-dihydropyrrolo[1,2-f][1,2,4]triazin-2-yl)ethylamino)pyrimidine-5-carbonitrile;
(S)-2-(1-(9H-purin-6-ylamino)ethyl)-5-(difluoromethyl)-3-phenylpyrrolo[1,2-f][1,2,4]triazin-4(3H)-one;
(S)-2-(1-(9H-purin-6-ylamino)ethyl)-3-phenylimidazo[1,2-f][1,2,4]triazin-4(3H)-one;
(S)-4-amino-6-(1-(4-oxo-3-phenyl-3,4-dihydroimidazo[1,2-f][1,2,4]triazin-2-yl)ethylamino)pyrimidine-5-carbonitrile;
2-(1-(9H-purin-6-ylamino)-3,3,3-trifluoropropyl)-3-phenylpyrrolo[1,2-f][1,2,4]triazin-4(3H)-one;
4-amino-6-(3,3,3-trifluoro-1-(4-oxo-3-phenyl-3,4-dihydropyrrolo[1,2-f][1,2,4]triazin-2-yl)propylamino)pyrimidine-5-carbonitrile;
(S)-4-amino-6-(2-(4-oxo-3-phenyl-3,4-dihydropyrrolo[1,2-f][1,2,4]triazin-2-yl)pyrrolidin-1-yl)pyrimidine-5-carbonitrile;
(S)-3-phenyl-2-(1-(pyrazolo[1,5-a]pyrimidin-7-ylamino)ethyl)pyrrolo[1,2-f][1,2,4]triazin-4(3H)-one;
2-((6-amino-9H-purin-9-yl)methyl)-5-(difluoromethyl)-3-phenylpyrrolo[1,2-f][1,2,4]triazin-4(3H)-one;
(S)-2-(1-(2-amino-9H-purin-6-yl)pyrrolidin-2-yl)-3-phenylpyrrolo[1,2-f][1,2,4]triazin-4(3H)-one;
(S)-2-(1-(4,6-diamino-1,3,5-triazin-2-ylamino)ethyl)-3-phenylpyrrolo[1,2-f][1,2,4]triazin-4(3H)-one;
(S)-2-((6-amino-9H-purin-9-yl)methyl)-5-chloro-3-(1-(5-fluoropyridin-2-yl)ethyl)pyrrolo[1,2-f][1,2,4]triazin-4(3H)-one;
(S)-2-(1-(2-amino-9H-purin-6-ylamino)ethyl)-3-(3,5-difluorophenyl)pyrrolo[1,2-f][1,2,4]triazin-4(3H)-one;
(S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile;
(S)-2-(1-(9H-purin-6-ylamino)ethyl)-4-oxo-3-phenyl-3,4-dihydropyrrolo[1,2-f][1,2,4]triazine-5-carbonitrile;
(R)-2-(1-(9H-purin-6-ylamino)-2-hydroxyethyl)-3-phenylpyrrolo[1,2-f][1,2,4]triazin-4(3H)-one;
(R)-4-amino-6-(2-hydroxy-1-(4-oxo-3-phenyl-3,4-dihydropyrrolo[1,2-f][1,2,4]triazin-2-yl)ethylamino)pyrimidine-5-carbonitrile;
(S)-2-(1-(2-amino-9H-purin-6-ylamino)ethyl)-3-phenylimidazo[1,2-f][1,2,4]triazin-4(3H)-one;
(S)-2-(1-(7H-pyrrolo[2,3-d]pyrimidin-4-ylamino)ethyl)-3-phenylpyrrolo[1,2-f][1,2,4]triazin-4(3H)-one;
(S)-4-amino-6-(methyl(1-(4-oxo-3-phenyl-3,4-dihydropyrrolo[1,2-f][1,2,4]triazin-2-yl)ethyl)amino)pyrimidine-5-carbonitrile;
(S)-2-(1-(methyl(9H-purin-6-yl)amino)ethyl)-3-phenylpyrrolo[1,2-f][1,2,4]triazin-4(3H)-one;
(S)-2-(1-(9H-purin-6-ylamino)ethyl)-5-methyl-3-phenylpyrrolo[1,2-f][1,2,4]triazin-4(3H)-one;
(S)-4-amino-6-(1-(5-methyl-4-oxo-3-phenyl-3,4-dihydropyrrolo[1,2-f][1,2,4]triazin-2-yl)ethylamino)pyrimidine-5-carbonitrile;
(S)-2-(1-(9H-purin-6-ylamino)ethyl)-7-methyl-3-phenylpyrrolo[1,2-f][1,2,4]triazin-4(3H)-one;
(S)-4-amino-6-(1-(7-methyl-4-oxo-3-phenyl-3,4-dihydropyrrolo[1,2-f][1,2,4]triazin-2-yl)ethylamino)pyrimidine-5-carbonitrile;
(S)-2-(4,4-difluoro-1-(9H-purin-6-yl)pyrrolidin-2-yl)-3-phenylpyrrolo[1,2-f][1,2,4]triazin-4(3H)-one;
(S)-4-amino-6-(4,4-difluoro-2-(4-oxo-3-phenyl-3,4-dihydropyrrolo[1,2-f][1,2,4]triazin-2-yl)pyrrolidin-1-yl)pyrimidine-5-carbonitrile;
(S)-2-(1-(9H-purin-6-ylamino)ethyl)-6-fluoro-3-phenylpyrrolo[1,2-f][1,2,4]triazin-4(3H)-one;
(S)-4-amino-6-(1-(6-fluoro-4-oxo-3-phenyl-3,4-dihydropyrrolo[1,2-f][1,2,4]triazin-2-yl)ethylamino)pyrimidine-5-carbonitrile;
2-((S)-1-(9H-purin-6-ylamino)ethyl)-3-((S)-1-phenylethyl)pyrrolo[1,2-f][1,2,4]triazin-4(3H)-one;
4-amino-6-((S)-1-(4-oxo-3-((S)-1-phenylethyl)-3,4-dihydropyrrolo[1,2-f][1,2,4]triazin-2-yl)ethylamino)pyrimidine-5-carbonitrile;
(S)-4-amino-6-(1-(3-(2,6-dimethylphenyl)-4-oxo-3,4-dihydropyrrolo[1,2-f][1,2,4]triazin-2-yl)ethylamino)pyrimidine-5-carbonitrile;
(S)-2-((9H-purin-6-ylamino)methyl)-3-(1-phenylethyl)pyrrolo[1,2-f][1,2,4]triazin-4(3H)-one;
(S)-4-amino-6-((4-oxo-3-(1-phenylethyl)-3,4-dihydropyrrolo[1,2-f][1,2,4]triazin-2-yl)methylamino)pyrimidine-5-carbonitrile;
(S)-2-(1-(5-fluoro-7H-pyrrolo[2,3-d]pyrimidin-4-ylamino)ethyl)-3-phenylpyrrolo[1,2-f][1,2,4]triazin-4(3H)-one;
(S)-2-(1-(9H-purin-6-ylamino)ethyl)-3-(2,6-dimethyl phenyl) pyrrolo[1,2-f][1,2,4]triazin-4(3H)-one;
(S)-4-amino-6-(1-(5-fluoro-4-oxo-3-phenyl-3,4-dihydropyrrolo[1,2-f][1,2,4]triazin-2-yl)ethylamino)pyrimidine-5-carbonitrile;
(S)-2-(1-(9H-purin-6-ylamino)ethyl)-5-fluoro-3-phenylpyrrolo[1,2-f][1,2,4]triazin-4(3H)-one;
(S)-2-(1-(6-amino-5-cyanopyrimidin-4-ylamino)ethyl)-3-(3,5-difluorophenyl)-4-oxo-3,4-dihydropyrrolo[1,2-f][1,2,4]triazine-5-carbonitrile;
(S)-4-amino-6-(1-(3-(3,5-difluorophenyl)-4-oxo-3,4-dihydroimidazo[1,2-f][1,2,4]triazin-2-yl)ethylamino)pyrimidine-5-carbonitrile;
(S)-2-(1-(9H-purin-6-ylamino)ethyl)-3-(3,5-difluorophenyl)-4-oxo-3,4-dihydropyrrolo[1,2-f][1,2,4]triazine-5-carbonitrile;
4-amino-6-((1S)-1-(5-(1,2-dihydroxyethyl)-4-oxo-3-phenyl-3,4-dihydropyrrolo[1,2-f][1,2,4]triazin-2-yl)ethylamino)pyrimidine-5-carbonitrile;
(S)-4-amino-6-(1-(3-(3,5-difluorophenyl)-4-oxo-5-(trifluoromethyl)-3,4-dihydropyrrolo[1,2-f][1,2,4]triazin-2-yl)ethylamino)pyrimidine-5-carbonitrile;
(S)-2-(1-(9H-Purin-6-ylamino)ethyl)-3-(3,5-difluorophenyl)-5-(trifluoromethyl)pyrrolo[1,2-f][1,2,4]triazin-4(3H)-one;
(S)-4-Amino-6-(1-(5-(hydroxymethyl)-4-oxo-3-phenyl-3,4-dihydropyrrolo[1,2-f][1,2,4]triazin-2-yl)ethylamino)pyrimidine-5-carbonitrile;
(S)-2-(1-(6-Amino-5-(trifluoromethyl)pyrimidin-4-ylamino)ethyl)-3-phenylpyrrolo[1,2-f][1,2,4]triazin-4(3H)-one;
(S)-2-(1-(6-Amino-5-cyanopyrimidin-4-ylamino)ethyl)-4-oxo-3-(pyridin-2-ylmethyl)-3,4-dihydropyrrolo[1,2-f][1,2,4]triazine-5-carbonitrile;
(S)-2-(1-(9H-Purin-6-ylamino)ethyl)-5-(difluoromethyl)-3-(3,5-difluorophenyl)pyrrolo[1,2-f][1,2,4]triazin-4(3H)-one;
(S)-2-(1-(9H-Purin-6-ylamino)ethyl)-3-(3,5-difluorophenyl)imidazo[1,2-f][1,2,4]triazin-4(3H)-one;
(S)-4-Amino-6-(1-(5-(difluoromethyl)-3-(3,5-difluorophenyl)-4-oxo-3,4-dihydropyrrolo[1,2-f][1,2,4]triazin-2-yl)ethylamino)pyrimidine-5-carbonitrile;
(S)-2-(1-(2-Amino-9H-purin-6-ylamino)ethyl)-5-(difluoromethyl)-3-(3,5-difluorophenyl)pyrrolo[1,2-f][1,2,4]triazin-4(3H)-one;
(S)-2-(1-(2-Amino-9H-purin-6-ylamino)ethyl)-3-(3,5-difluorophenyl)-4-oxo-3,4-dihydropyrrolo[1,2-f][1,2,4]triazine-5-carbonitrile;
2-(1-(9H-Purin-6-ylamino)-2,2,2-trifluoroethyl)-3-phenylpyrrolo[1,2-f][1,2,4]triazin-4(3H)-one;
(S)-4-Amino-6-(1-(3-benzyl-4-oxo-3,4-dihydropyrrolo[1,2-f][1,2,4]triazin-2-yl)ethylamino)pyrimidine-5-carbonitrile;
(S)-2-(1-(6-Amino-5-fluoropyrimidin-4-ylamino)ethyl)-3-phenylpyrrolo[1,2-f][1,2,4]triazin-4(3H)-one;
(S)-2-(1-(6-Amino-5-fluoropyrimidin-4-ylamino)ethyl)-5-(difluoromethyl)-3-(3,5-difluorophenyl)pyrrolo[1,2-f][1,2,4]triazin-4(3H)-one;
(S)-2-(1-(6-Amino-5-cyanopyrimidin-4-ylamino)propyl)-3-(3,5-difluorophenyl)-4-oxo-3,4-dihydropyrrolo[1,2-f][1,2,4]triazine-5-carbonitrile;
(S)-2-(1-(6-Amino-5-cyanopyrimidin-4-ylamino)ethyl)-3-(3,5-dichlorophenyl)-4-oxo-3,4-dihydropyrrolo[1,2-f][1,2,4]triazine-5-carbonitrile;
(S)-2-(1-(6-Amino-5-fluoropyrimidin-4-ylamino)ethyl)-3-(3,5-difluorophenyl)-4-oxo-3,4-dihydropyrrolo[1,2-f][1,2,4]triazine-5-carbonitrile;
(S)-2-(1-(6-Amino-5-(trifluoromethyl)pyrimidin-4-ylamino)ethyl)-4-oxo-3-phenyl-3,4-dihydropyrrolo[1,2-f][1,2,4]triazine-5-carbonitrile;
(R)-2-(1-(6-Amino-5-cyanopyrimidin-4-ylamino)-2-hydroxyethyl)-3-(3,5-difluorophenyl)-4-oxo-3,4-dihydropyrrolo[1,2-f][1,2,4]triazine-5-carbonitrile;
(S)-2-(1-(6-Amino-5-carbamoylpyrimidin-4-ylamino)ethyl)-3-(3,5-difluorophenyl)-4-oxo-3,4-dihydropyrrolo[1,2-f][1,2,4]triazine-5-carboxamide;
(S)-2-(1-(6-Amino-5-cyanopyrimidin-4-ylamino)ethyl)-3-(3,5-difluorophenyl)-4-oxo-3,4-dihydropyrrolo[1,2-f][1,2,4]triazine-5-carboxamide;
(S)-2-(1-(6-Amino-5-cyanopyrimidin-4-ylamino)ethyl)-3-(2-chlorobenzyl)-4-oxo-3,4-dihydropyrrolo[1,2-f][1,2,4]triazine-5-carbonitrile;
2-((S)-1-(6-amino-5-cyanopyrimidin-4-ylamino)ethyl)-4-oxo-3-((S)-tetrahydro-2H-pyran-3-yl)-3,4-dihydropyrrolo[1,2-f][1,2,4]triazine-5-carbonitrile;
(R)-4-Amino-6-(1-(3-(3,5-difluorophenyl)-4-oxo-5-(trifluoromethyl)-3,4-dihydropyrrolo[1,2-f][1,2,4]triazin-2-yl)-2-hydroxyethylamino)pyrimidine-5-carbonitrile;
(S)-2-(1-(2-Amino-5-fluoropyrimidin-4-ylamino)ethyl)-3-(3,5-difluorophenyl)-4-oxo-3,4-dihydropyrrolo[1,2-f][1,2,4]triazine-5-carbonitrile;
(S)-2-(1-(2-Amino-5-cyanopyrimidin-4-ylamino)ethyl)-3-(3,5-difluorophenyl)-4-oxo-3,4-dihydropyrrolo[1,2-f][1,2,4]triazine-5-carbonitrile;
((S)-2-(1-(9H-Purin-6-ylamino)ethyl)-3-(3,5-difluorophenyl)-5-(2H-tetrazol-5-yl)pyrrolo[1,2-f][1,2,4]triazin-4(3H)-one;
(S)-4-Amino-6-(1-(3-((5-methylisoxazol-3-yl)methyl)-4-oxo-3,4-dihydropyrrolo[1,2-f][1,2,4]triazin-2-yl)ethylamino)pyrimidine-5-carbonitrile;
(S)-4-Amino-6-(1-(4-oxo-3-phenyl-7-(trifluoromethyl)-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-4-oxo-3-phenyl-3,4-dihydropyrrolo[1,2-f][1,2,4]triazine-7-carbonitrile;
(S)-2-(1-(6-Amino-5-cyanopyrimidin-4-ylamino)ethyl)-3-(1-(4-methoxybenzyl)-1H-pyrazol-4-yl)-4-oxo-3,4-dihydropyrrolo[1,2-f][1,2,4]triazine-5-carbonitrile;
(S)-4-amino-6-(1-(4-oxo-3-phenyl-5-(thiazol-2-yl)-3,4-dihydropyrrolo[1,2-f][1,2,4]triazin-2-yl)ethylamino)pyrimidine-5-carbonitrile;
(S)-2-(1-(2,6-Diamino-5-cyanopyrimidin-4-ylamino)ethyl)-3-(3,5-difluorophenyl)-4-oxo-3,4-dihydropyrrolo[1,2-f][1,2,4]triazine-5-carbonitrile;
(S)-4-Amino-6-(1-(5-(morpholinomethyl)-4-oxo-3-phenyl-3,4-dihydropyrrolo[1,2-f][1,2,4]triazin-2-yl)ethylamino)pyrimidine-5-carbonitrile;
2-((S)-1-(6-Amino-5-cyanopyrimidin-4-ylamino)ethyl)-4-oxo-3-((R)-1-phenylethyl)-3,4-dihydropyrrolo[1,2-f][1,2,4]triazine-5-carbonitrile;
(S)-4-Amino-6-(1-(4-oxo-3-(1H-pyrazol-4-yl)-3,4-dihydropyrrolo[1,2-f][1,2,4]triazin-2-yl)ethylamino)pyrimidine-5-carbonitrile;
(S)-2-(1-(9H-Purin-6-ylamino)ethyl)-3-(3,5-difluorophenyl)-5-(5-methyl-1,2,4-oxadiazol-3-yl)pyrrolo[1,2-f][1,2,4]triazin-4(3H)-one;
4-amino-6-((S)-1-(4-oxo-3-((S)-tetrahydro-2H-pyran-3-yl)-5-(trifluoromethyl)-3,4-dihydropyrrolo[1,2-f][1,2,4]triazin-2-yl)ethylamino)pyrimidine-5-carbonitrile;
(S)-4-Amino-6-(1-(3-(5-methyl-1H-pyrazol-3-yl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-4-oxo-3-phenyl-3,4-dihydropyrrolo[1,2-f][1,2,4]triazine-5-carboxylic acid;
2-((S)-1-(6-amino-5-cyanopyrimidin-4-ylamino)ethyl)-4-oxo-3-((R)-tetrahydro-2H-pyran-3-yl)-3,4-dihydropyrrolo[1,2-f][1,2,4]triazine-5-carbonitrile;
(S)-4-Amino-6-(1-(3-(5-fluoropyridin-3-yl)-4-oxo-3,4-dihydropyrrolo[1,2-f][1,2,4]triazin-2-yl)ethylamino)pyrimidine-5-carbonitrile;
(S)-4-Amino-6-(1-(4-oxo-3-(1H-pyrazol-3-yl)-3,4-dihydropyrrolo[1,2-f][1,2,4]triazin-2-yl)ethylamino)pyrimidine-5-carbonitrile;
(S)-4-Amino-6-(1-(4-oxo-3-(pyrimidin-5-yl)-3,4-dihydropyrrolo[1,2-f][1,2,4]triazin-2-yl)ethylamino)pyrimidine-5-carbonitrile;
4-amino-6-((S)-1-(4-oxo-3-((R)-tetrahydro-2H-pyran-3-yl)-5-(trifluoromethyl)-3,4-dihydropyrrolo[1,2-f][1,2,4]triazin-2-yl)ethylamino)pyrimidine-5-carbonitrile;
(S)-2,4-Diamino-6-(1-(4-oxo-3-phenyl-3,4-dihydropyrrolo[1,2-f][1,2,4]triazin-2-yl)ethylamino)pyrimidine-5-carbonitrile;
(S)-4-(1-(3-((1H-Pyrazol-3-yl)methyl)-4-oxo-3,4-dihydropyrrolo[1,2-f][1,2,4]triazin-2-yl)ethylamino)-6-aminopyrimidine-5-carbonitrile;
(S)-4-Amino-6-(1-(4-oxo-3-(tetrahydro-2H-pyran-4-yl)-3,4-dihydropyrrolo[1,2-f][1,2,4]triazin-2-yl)ethylamino)pyrimidine-5-carbonitrile;
(S)-4-Amino-6-(1-(4-oxo-3-(2,2,2-trifluoroethyl)-3,4-dihydropyrrolo[1,2-f][1,2,4]triazin-2-yl)ethylamino)pyrimidine-5-carbonitrile;
(S)-4-Amino-6-(1-(3-cyclobutyl-4-oxo-3,4-dihydropyrrolo[1,2-f][1,2,4]triazin-2-yl)ethylamino)pyrimidine-5-carbonitrile;
(S)-2-Amino-4-(1-(4-oxo-3-phenyl-3,4-dihydropyrrolo[1,2-f][1,2,4]triazin-2-yl)ethylamino)pyrimidine-5-carbonitrile;
4-Amino-6-(1-(5-(1-methyl-1H-pyrazol-4-yl)-4-oxo-3-phenyl-3,4-dihydropyrrolo[1,2-f][1,2,4]triazin-2-yl)ethylamino)pyrimidine-5-carbonitrile;
(S)-4-Amino-6-(1-(3-cyclopropyl-4-oxo-3,4-dihydropyrrolo[1,2-f][1,2,4]triazin-2-yl)ethylamino)pyrimidine-5-carbonitrile;
(S)-4-Amino-6-(1-(5-bromo-4-oxo-3,4-dihydropyrrolo[1,2-f][1,2,4]triazin-2-yl)ethylamino)pyrimidine-5-carbonitrile;
4-amino-6-((S)-1-(4-oxo-3-((R)-tetrahydro-2H-pyran-3-yl)-3,4-dihydropyrrolo[1,2-f][1,2,4]triazin-2-yl)ethylamino)pyrimidine-5-carbonitrile;
(S)-4-Amino-6-(1-(5-bromo-4-oxo-3-phenyl-3,4-dihydropyrrolo[1,2-f][1,2,4]triazin-2-yl)ethylamino)pyrimidine-5-carbonitrile;
2-((3-Iodo-1H-pyrazolo[3,4-d]pyrimidin-4-ylamino)methyl)-5-methyl-3-o-tolylpyrrolo[1,2-f][1,2,4]triazin-4(3H)-one;
(S)-2-(1-(6-Amino-5-cyanopyrimidin-4-ylamino)ethyl)-3-(5-fluoropyridin-3-yl)-4-oxo-3,4-dihydropyrrolo[1,2-f][1,2,4]triazine-5-carbonitrile;
4-amino-6-((S)-1-(4-oxo-3-((S)-tetrahydro-2H-pyran-3-yl)-3,4-dihydropyrrolo[1,2-f][1,2,4]triazin-2-yl)ethylamino)pyrimidine-5-carbonitrile;
(S)-4-Amino-6-(1-(4-oxo-3-phenyl-5-(1H-pyrazol-4-yl)-3,4-dihydropyrrolo[1,2-f][1,2,4]triazin-2-yl)ethylamino)pyrimidine-5-carbonitrile;
(S)-4-Amino-6-(1-(3-(isoxazol-3-yl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-N,N-dimethyl-4-oxo-3-phenyl-3,4-dihydropyrrolo[1,2-f][1,2,4]triazine-5-carboxamide;
(S)-4-Amino-6-(1-(3-(1-methyl-1H-pyrazol-3-yl)-4-oxo-3,4-dihydropyrrolo[1,2-f][1,2,4]triazin-2-yl)ethylamino)pyrimidine-5-carbonitrile
(S)-2-(1-(6-Amino-5-cyanopyrimidin-4-ylamino)ethyl)-4-oxo-3-phenyl-N-propyl-3,4-dihydropyrrolo[1,2-f][1,2,4]triazine-5-carboxamide;
2-((S)-1-(9H-Purin-6-ylamino)ethyl)-3-(tetrahydro-2H-pyran-3-yl)pyrrolo[1,2-f][1,2,4]triazin-4(3H)-one;
2-((S)-1-(9H-purin-6-ylamino)ethyl)-3-((S)-tetrahydro-2H-pyran-3-yl)pyrrolo[1,2-f][1,2,4]triazin-4(3H)-one;
(S)-4-Amino-6-(3-hydroxy-1-(4-oxo-3-phenyl-3,4-dihydropyrrolo[1,2-f][1,2,4]triazin-2-yl)propylamino)pyrimidine-5-carbonitrile;
(S)-2-(1-(9H-Purin-6-ylamino)-3-hydroxypropyl)-3-phenylpyrrolo[1,2-f][1,2,4]triazin-4(3H)-one;
(R)-4-Amino-6-(1-(3-(3,5-difluorophenyl)-4-oxo-3,4-dihydropyrrolo[1,2-f][1,2,4]triazin-2-yl)-2-hydroxyethylamino)pyrimidine-5-carbonitrile;
4-Amino-6-((4-oxo-3-o-tolyl-3,4-dihydropyrrolo[1,2-f][1,2,4]triazin-2-yl)methylamino)pyrimidine-5-carbonitrile;
(S)-4-Amino-6-(1-(5-(2-hydroxyethyl)-4-oxo-3-phenyl-3,4-dihydropyrrolo[1,2-f][1,2,4]triazin-2-yl)ethylamino)pyrimidine-5-carbonitrile;
S)-2-(1-(6-amino-5-cyanopyrimidin-4-ylamino)-3-hydroxypropyl)-3-(3,5-difluorophenyl)-4-oxo-3,4-dihydropyrrolo[1,2-f][1,2,4]triazine-5-carbonitrile;
(S)-2-(1-(9H-Purin-6-ylamino)ethyl)-3-(5-fluoropyridin-3-yl)-4-oxo-3,4-dihydropyrrolo[1,2-f][1,2,4]triazine-5-carbonitrile;
(S)-4-Amino-6-(1-(5-(2-methyloxazol-5-yl)-4-oxo-3-phenyl-3,4-dihydropyrrolo[1,2-f][1,2,4]triazin-2-yl)ethylamino)pyrimidine-5-carbonitrile;
(S)-4-Amino-6-(1-(5-(2-methoxyethyl)-4-oxo-3-phenyl-3,4-di hydropyrrolo[1,2-f][1,2,4]triazin-2-yl)ethylamino)pyrimidine-5-carbonitrile;
(S)-Propyl 2-(1-(6-amino-5-cyanopyrimidin-4-ylamino)ethyl)-4-oxo-3-phenyl-3,4-dihydropyrrolo[1,2-f][1,2,4]triazine-5-carboxylate;
(S)-4-Amino-6-(3-hydroxy-1-(4-oxo-3,4-dihydropyrrolo[1,2-f][1,2,4]triazin-2-yl)propylamino)pyrimidine-5-carbonitrile;
(S)-2-(1-(9H-Purin-6-ylamino)-3-hydroxypropyl)pyrrolo[1,2-f][1,2,4]triazin-4(3H)-one;
(S)-4-amino-6-(1-(3-(3,5-difluorophenyl)-4-oxo-5-(trifluoromethyl)-3,4-dihydropyrrolo[1,2-f][1,2,4]triazin-2-yl)-3-hydroxypropylamino)pyrimidine-5-carbonitrile;
(S)-4-Amino-6-(1-(4-oxo-3-(6-(trifluoromethyl)pyridin-2-yl)-3,4-dihydropyrrolo[1,2-f][1,2,4]triazin-2-yl)ethylamino)pyrimidine-5-carbonitrile;
(S)-4-Amino-6-(1-(5-bromo-4-oxo-3-(3-(trifluoromethyl)phenyl)-3,4-dihydropyrrolo[1,2-f][1,2,4]triazin-2-yl)ethylamino)pyrimidine-5-carbonitrile;
(S)-2-(2-(1-(6-Amino-5-cyanopyrimidin-4-ylamino)ethyl)-4-oxo-3-phenyl-3,4-dihydropyrrolo[1,2-f][1,2,4]triazin-5-yl)ethyl acetate;
(S)-2-(1-(9H-Purin-6-ylamino)ethyl)-3-(6-(trifluoromethyl)pyridin-2-yl)pyrrolo[1,2-f][1,2,4]triazin-4(3H)-one;
2-((2S,4R)-1-(6-Amino-5-cyanopyrimidin-4-yl)-4-hydroxypyrrolidin-2-yl)-3-(3,5-difluorophenyl)-4-oxo-3,4-dihydropyrrolo[1,2-f][1,2,4]triazine-5-carbonitrile;
4-Amino-6-((2S,4R)-2-(5-(aminomethyl)-3-(3,5-difluorophenyl)-4-oxo-3,4-dihydropyrrolo[1,2-f][1,2,4]triazin-2-yl)-4-hydroxypyrrolidin-1-yl)pyrimidine-5-carbonitrile;
(S)-4-Amino-6-(1-(5-(4-methyl-1H-imidazol-1-yl)-4-oxo-3-phenyl-3,4-dihydropyrrolo[1,2-f][1,2,4]triazin-2-yl)ethylamino)pyrimidine-5-carbonitrile;
(S)-4-Amino-6-(1-(5-bromo-3-(3-methoxyphenyl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-4-oxo-3-(3-(trifluoromethyl)phenyl)-3,4-dihydropyrrolo[1,2-f][1,2,4]triazine-5-carbonitrile;
(S)-4-Amino-6-(1-(5-bromo-3-(3-hydroxyphenyl)-4-oxo-3,4-dihydropyrrolo[1,2-f][1,2,4]triazin-2-yl)ethylamino)pyrimidine-5-carbonitrile;
(S)-4-Amino-6-(1-(3-(3-methoxyphenyl)-4-oxo-3,4-dihydropyrrolo[1,2-f][1,2,4]triazin-2-yl)ethylamino)pyrimidine-5-carbonitrile;
(S)-4-Amino-6-(1-(3-(3-hydroxyphenyl)-4-oxo-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-3-(3-methoxyphenyl)-4-oxo-3,4-dihydropyrrolo[1,2-f][1,2,4]triazine-5-carbonitrile;
4-Amino-6-(1-(4-oxo-3-phenyl-3,4-dihydropyrrolo[1,2-f][1,2,4]triazin-2-yl)cyclopropylamino)pyrimidine-5-carbonitrile;
2-(1-(9H-Purin-6-ylamino)cyclopropyl)-3-phenylpyrrolo[1,2-f][1,2,4]triazin-4(3H)-one;
(S)-4-Amino-6-(1-(4-oxo-3-(3-(trifluoromethyl)phenyl)-3,4-dihydropyrrolo[1,2-f][1,2,4]triazin-2-yl)ethylamino)pyrimidine-5-carbonitrile;
(S)-2-(1-(6-Amino-5-cyanopyrimidin-4-ylamino)ethyl)-3-(3-hydroxyphenyl)-4-oxo-3,4-dihydropyrrolo[1,2-f][1,2,4]triazine-5-carbonitrile;
(S)-2-(1-(9H-purin-6-ylamino)ethyl)-3-(pyridin-2-yl)pyrrolo[1,2-f][1,2,4]triazin-4(3H)-one;
(S)-2-(1-(9H-purin-6-ylamino)propyl)-3-phenylimidazo[1,2-f][1,2,4]triazin-4(3H)-one; and
(S)-4-amino-6-(1-(4-oxo-3-phenyl-3,4-dihydroimidazo[1,2-f][1,2,4]triazin-2-yl)propylamino)pyrimidine-5-carbonitrile.

Further preferred inhibitors of phosphoinositide 3-kinase delta may be selected from nortriptyline, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib, for instance idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib.

Typically, inhibitors of phosphoinositide 3-kinase delta for use in accordance with the present invention are selected from the group consisting of nortriptyline, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib, for instance idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179 and panulisib.

Preferably, inhibitors of phosphoinositide 3-kinase delta for use in accordance with the present invention are selected from the group consisting of nortriptyline, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008 and CLR-457, for instance idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, LAS191954, XL-499, KAR-4141, RP-5090, PWT-143, IPI-443, RP-6503, ONO-146040, SPR-965, LOR-220, SF-2626, X-339, X-480, PQR-401, INCB-050465, LS-008 and CLR-457.

More preferably, inhibitors of phosphoinositide 3-kinase delta for use in accordance with the present invention are selected from the group consisting of nortriptyline, idelalisib, duvelisib, enzastaurin, rigosertib, GSK-2269557, UCB-5857, RV-1729, RP-6530, LAS191954, XL-499, KAR-4141, RP-5090, PWT-143, IPI-443 and RP-6503, for instance idelalisib, duvelisib, enzastaurin, rigosertib, GSK-2269557, UCB-5857, RV-1729, RP-6530, LAS191954, XL-499, KAR-4141, RP-5090, PWT-143, IPI-443 and RP-6503.

Still more preferably, inhibitors of phosphoinositide 3-kinase delta for use in accordance with the present invention are selected from the group consisting of idelalisib, duvelisib, UCB-5857, RP-6530, LAS191954, XL-499, KAR-4141, RP-5090, PWT-143, IPI-443 and RP-6503.

Alternatively, inhibitors of phosphoinositide 3-kinase delta for use in accordance with the present invention are selected from the group consisting of LAS191954, alpelisib ((S)—N1-(4-methyl-5-(2-(1,1,1-trifluoro-2-methylpropan-2-yl)pyridin-4-yl)thiazol-2-yl)pyrrolidine-1,2-dicarboxamide), duvelisib ((S)-3-(1-((9H-purin-6-yl)amino)ethyl)-8-chloro-2-phenylisoquinolin-1 (2H)-one), rigosertib sodium (sodium (E)-2-((2-methoxy-5-(((2,4,6-trimethoxystyryl)sulfonyl)methyl)phenyl)amino)acetate), and 6-(2-((4-amino-3-(3-hydroxyphenyl)-1H-pyrazolo[3,4-d]pyrimidin-1-yl)methyl)-3-(2-chlorobenzyl)-4-oxo-3,4-dihydroquinazolin-5-yl)-N, N-bis(2-methoxyethyl)hex-5-ynamide.

Most preferably, the inhibitor of phosphoinositide 3-kinase delta for use in accordance with the present invention is LAS191954.

LAS191954, which has the structure of formula (A) and corresponds to (S)-2-(1-(6-amino-5-cyanopyrimidin-4-ylamino)ethyl)-4-oxo-3-phenyl-3,4-dihydropyrrolo[1,2-f][1,2,4]triazine-5-carbonitrile, as well as a process for its manufacture, is described in the International Patent Application No. WO 2012/146666.

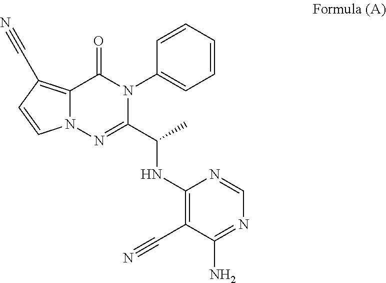 [SMILES: C[C@H](Nc1ncnc(N)c1C#N)c1nn2ccc(C#N)c2c(=O)n1-c1ccccc1]

In a preferred embodiment, the compound is a pharmaceutically acceptable crystalline addition salt of LAS191954 with a sulfonic acid derivative selected from methanesulfonic acid, naphthalene-2-sulfonic acid and para-toluenesulfonic acid, or a pharmaceutically acceptable solvate thereof.

In a particular embodiment, the compound is LAS191954 methanesulfonate, or a pharmaceutically acceptable solvate thereof.

Typically, methanesulfonic acid (CAS RN 75-75-2) is a colourless liquid with the molecular formula CH₄O₃S (molecular weight of 96.11 g/mol). Salts of methanesulfonic acid are known as methanesulfonates, mesilates (International Nonproprietary Name or INN) or mesylates (United States Adopted Name or USAN).

In another particular embodiment, the compound is LAS191954 naphthalene-2-sulfonate, or a pharmaceutically acceptable solvate thereof.

Typically, naphthalene-2-sulfonic acid (CAS RN 120-18-3) is a solid at 20° C. with the molecular formula C₁₀H₈O₃S (molecular weight of 208.24 g/mol). Salts of naphthalene-2-sulfonic acid are known as naphthalene-2-sulfonates, napsilates (INN) or napsylates (USAN).

In another particular embodiment, the compound is LAS191954 para-toluenesulfonate, or a pharmaceutically acceptable solvate thereof.

Typically, para-toluenesulfonic acid (CAS RN 104-15-4) or tosylic acid is a solid at 20° C. with the molecular formula C₇H₈O₃S (molecular weight of 172.20 g/mol). Salts of para-toluenesulfonic acid are known as para-toluenesulfonates, tosilates (INN) or tosylates (USAN).

In still another particular embodiment, the compound is LAS191954, para-toluenesulfonate monohydrate.

Compounds for use in the methods of the present invention are typically commercially available or may be prepared in accordance with known methods.

The immunobullous skin diseases treated in accordance with the present invention are characterized by pathogenic autoantibodies directed at antigens whose function is either cell-to-cell adhesion within the epidermis or adhesion of stratified squamous epithelium to dermis or mesenchyme. These target antigens are components of desmosomes or the functional unit of the basement membrane zone known as the adhesion complex (see Rook's Textbook of Dermatology, Wiley-Blackwell, Chapter 40—Immunobullous diseases).

Typically, the immunobullous skin disease is mediated by anti-Dsg autoantibodies. Preferably, the immunobullous skin disease is mediated by anti-Dsg1 and/or anti-Dsg3 autoantibodies. More preferably, the immunobullous skin disease is mediated by anti-Dsg3 autoantibodies.

Typically, the immunobullous skin disease is mediated by anti-dsDNA autoantibodies.

The immunobullous skin disease may be mediated by both anti-dsDNA autoantibodies and anti-Dsg autoantibodies as defined above.

Immunobullous skin diseases mediated by autoantibodies which may be treated in accordance with the invention include (but are not limited to):
Intraepidermal immunobullous diseases, such as pemphigus vulgaris, pemphigus vegetans, pemphigus foliaceus, endemic pemphigus foliaceus, intercellular IgA dermatosis, paraneoplastic pemphigus; and
Subepidermal immunobullous diseases, such bullous pemphigoid, mucous membrane pemphigoid, pemphigoid gestationis, linear IgA disease, epidermolysis bullosa acquisita, bullous systemic lupus erythematosus and dermatitis herpetiformis.

Typically, the immunobullous skin disease mediated by autoantibodies is pemphigus vulgaris, pemphigus vegetans, pemphigus foliaceus, endemic pemphigus foliaceus, paraneoplastic pemphigus or epidermolysis bullosa acquisita.

Usually, the immunobullous skin disease mediated by autoantibodies is pemphigus vulgaris, pemphigus vegetans, pemphigus foliaceus, endemic pemphigus foliaceus, or paraneoplastic pemphigus.

In some circumstances, the immunobullous skin disease mediated by autoantibodies is pemphigus vulgaris, pemphigus foliaceus or epidermolysis bullosa acquisita.

Preferably, the immunobullous skin disease mediated by autoantibodies is pemphigus vulgaris or pemphigus foliaceus.

More preferably, the immunobullous skin disease mediated by autoantibodies is pemphigus vulgaris or epidermolysis bullosa acquisita.

Most preferably, the immunobullous skin disease mediated by autoantibodies is pemphigus vulgaris.

In a preferred embodiment, the compound is LAS191954 or a pharmaceutically acceptable salt and/or solvate thereof and the immunobullous skin disease mediated by autoantibodies is pemphigus vulgaris.

In a more preferred embodiment, the compound is LAS191954 methanesulfonate, or a pharmaceutically acceptable solvate thereof and the immunobullous skin disease mediated by autoantibodies is pemphigus vulgaris.

In another more preferred embodiment, the compound is LAS191954 naphthalene-2-sulfonate, or a pharmaceutically acceptable solvate thereof and the immunobullous skin disease mediated by autoantibodies is pemphigus vulgaris.

In another more preferred embodiment, the compound is LAS191954para-toluenesulfonate, or a pharmaceutically acceptable solvate thereof and the immunobullous skin disease mediated by autoantibodies is pemphigus vulgaris.

In another more preferred embodiment, the compound is LAS191954 para-toluenesulfonate monohydrate and the immunobullous skin disease mediated by autoantibodies is pemphigus vulgaris.

Typically, the compounds as defined herein are for use in the treatment of an immunobullous skin disease by oral administration in a human or animal patient, preferably a human, canine, feline or equine patient, more preferably a human patient.

As discussed above, treatment of immunobullous skin diseases mediated by autoantibodies with phosphoinositide 3-Kinase delta (PI3K delta) inhibitors advantageously targets B-lymphocyte functions and reduces pathogenic IgG antibody titers against autoantigens associated with such diseases, specifically reducing the production of antibodies to Dsg3, which are associated with immunobullous skin diseases.

Thus, typically, the compounds as defined herein are for use in the treatment of an immunobullous skin disease mediated by autoantibodies as defined herein by oral administration by one or more of:
prevention of B lymphocyte formation; and/or
attenuation of B cell function; and/or
reduction of the production of antibodies, typically antibodies to Dsg, preferably antibodies to Dsg3; and/or
reduction in the titer of autoantibodies, typically antibodies to Dsg, preferably antibodies to Dsg3.

The present invention also provides a compound as defined herein for use in prevention of B lymphocyte formation in a mammal, typically a human, suffering from an immunobullous skin disease mediated by autoantibodies as defined herein by oral administration.

The present invention also provides a compound as defined herein for use in attenuation of B cell function in a mammal, typically a human, suffering from an immunobullous skin disease mediated by autoantibodies as defined herein by oral administration.

The present invention also provides a compound as defined herein for use in reduction of the production of antibodies, typically antibodies to Dsg in a mammal, typically a human, suffering from an immunobullous skin disease mediated by autoantibodies as defined herein by oral administration. Preferably, the present invention provides a compound as defined herein for use in reduction of the production of antibodies to Dsg3 in a mammal, typically a human, suffering from an immunobullous skin disease mediated by autoantibodies as defined herein by oral administration.

The present invention also provides a compound as defined herein for use in reduction in the titer of autoantibodies, typically antibodies to Dsg in a mammal, typically a human, suffering from an immunobullous skin disease mediated by autoantibodies by oral administration. Preferably, the present invention provides a compound as defined herein for use in reduction in the titer of antibodies to Dsg3 in a mammal, typically a human, suffering from an immunobullous skin disease mediated by autoantibodies as defined herein by oral administration.

The compound for use in the method of the present invention may be co-administered with a therapeutically effective amount of one or more other therapeutic agents useful in the treatment or prevention of immunobullous skin diseases mediated by autoantibodies as defined herein.
a) The other therapeutic agent may be chosen from the group consisting of Immunosuppressants, such as Imuran (azathioprine), cyclophosphamide, sirolimus or Purinethol (6-mercaptopurine or 6-MP);
b) Corticoids and glucocorticoids such as prednisolone, prednisone, methylprednisolone, fluticasone, dexamethasone, mometasone, budesonide, ciclesonide or beta-metasone, for instance prednisone, methylprednisolone, fluticasone, dexamethasone, mometasone, budesonide, ciclesonide or beta-metasone;
c) Anti-CD20 (lymphocyte protein) monoclonal antibodies such as Rituximab, Ocrelizumab, Ofatumumab or TRU-015;
d) Anti-CD52 (lymphocyte protein) monoclonal antibodies such as alemtuzumab;
e) Anti-CD25 (lymphocyte protein) such as daclizumab;
f) Anti-CD88 (lymphocyte protein), such as eculizumab or pexilizumab;
g) Anti-Interleukin 6 Receptor (IL-6R), such as tocilizumab;
h) Anti-Interleukin 12 Receptor (IL-12R)/Interleukin 23 Receptor (IL-23R), such as ustekinumab;
i) Anti-BAFF/BlyS such as belimumab, tabalumab, or blisibimod
j) Anti-TACl such as atacicept
k) Anti-BAFF receptor such as VAY736
l) Anti-CD19 such as MEDI-551
m) Anti-ICOSL such as AMG-557
n) Anti-FasL monoclonal antibodies
o) Btk inhibitors like ibrutinib
p) Calcineurin inhibitors such as cyclosporin A, pimecrolimus or tacrolimus;
q) Dihydrofolate reductase inhibitors, such as Methotrexate or CH-1504;
r) Dihydroorotate dehydrogenase (DHODH) inhibitors such as leflunomide or teriflunomide;
s) Immunomodulators such as Glatiramer acetate (Copaxone), Laquinimod or Imiquimod;
t) Inhibitors of DNA synthesis and repair, such as Mitoxantrone or Cladribine;
u) Anti-alpha 4 integrin antibodies, such as Natalizumab (Tysabri);
v) Alpha 4 integrin antagonists such as R-1295, TBC-4746, CDP-323, ELND-002, Firategrast or TMC-2003;
w) Fumaric acid esters, such as dimethyl fumarate;
x) Anti-tumor necrosis factor-alpha (Anti-TNF-alpha) monoclonal antibodies such as Infliximab, Adalimumab or Certolizumab pegol;
y) Soluble Tumor necrosis factor-alpha (TNF-alpha) Antagonists such as Etanercept;
z) Inosine-monophosphate dehydrogenase (IMPDH) inhibitors, such as mycophenolate mophetyl, ribavirin, mizoribine or mycophenolic acid;
aa) Cannabinoid receptor agonists such as Sativex;
bb) Chemokine CCR1 antagonists such as MLN-3897 or PS-031291;
cc) Chemokine CCR2 antagonists such as INCB-8696;
dd) Nuclear factor-kappaB (NF-kappaB or NFKB) Activation Inhibitors such as Sulfasalazine, Iguratimod or MLN-0415;
ee) Adenosine A_{2A} agonists, such as ATL-313, ATL-146, CGS-21680, Regadenoson or UK-432,097;
ff) Sphingosine-1 (S1P) phosphate receptor agonists such as fingolimod, BAF-312, or ACT128800;
gg) Sphingosine-1 (S1P) liase inhibitors such as LX2931;
hh) Spleen tyrosine kinase (Syk) inhibitors, such as R-112;
ii) Protein Kinase Inhibitors (PKC) inhibitors, such as NVP-AEB071;
jj) Histamine 1 (H1) receptor antagonists, such as azelastine or ebastine;
kk) Mast cell stabilizers, such as nedocromil or chromoglycate;
ll) Chemoattractant receptor homologous molecule expressed on TH₂ cells (CRTH2) inhibitors, such as OC-459, AZD-1981, ACT-129968, QAV-680;
mm) Vitamin D derivatives like calcipotriol (Daivonex);
nn) Anti-inflammatory agents, such as non-steroidal anti-inflammatory drugs (NSAIDs) or selective cyclooxygenase-2 (COX-2) inhibitors such as aceclofenac, diclofenac, ibuprofen, naproxen, apricoxib, celecoxib, cimicoxib, deracoxib, etoricoxib, lumiracoxib, parecoxib sodium, rofecoxib, selenocoxib-1 or valdecoxib;
oo) Anti-viral agents, such as aciclovir or tenofovir;
pp) Phosphodiestearase (PDE) III inhibitors;
qq) Phosphosdiesterase (PDE) IV inhibitors such as roflumilast or GRC-4039;
rr) Dual Phosphodiestearase (PDE) Ill/IV inhibitors;
ss) p38 Mitogen-Activated Protein Kinase (p38 MAPK) Inhibitors such as ARRY-797;
tt) Mitogen-activated extracellular signal regulated kinase kinase (MEK) inhibitor, such as ARRY-142886 or ARRY-438162;
uu) Janus kinase (JAK) inhibitors, such as tofacitinib (previously known as tasocitinib or CP-690,550) or INCB-18424;
vv) Interferons comprising Interferon beta 1a such as Avonex from Biogen Idec, CinnoVex from CinnaGen and Rebif from EMD Serono, and Interferon beta 1b such as Betaferon from Schering and Betaseron from Berlex;
ww) Interferon alpha such as Sumiferon MP;
xx) Epidermal Growth Factor Receptor (EGFR) inhibitors such as erlotinib, Trastuzumab, Herceptin, Avastin, Platins (cisplatin, carboplatin) or Temazolamide;
yy) Antineoplastic agents such as Docetaxel, Estramustine, Anthracyclines, (doxorubicin (Adriamycin), epirubicin (Ellence), and liposomal doxorubicin (Doxil)), Taxanes (docetaxel (Taxotere), paclitaxel (Taxol), and protein-bound paclitaxel (Abraxane)), Cyclophosphamide (Cytoxan), Capecitabine (Xeloda), 5-fluorouracil (5-FU), Gemcitabine (Gemzar) or Vinorelbine (Navelbine);
zz) Tetracyclines, such as methacycline, doxycycline or minocycline;
aaa) Analgesics, such as paracetamol;
bbb) Opioids such as, morphine, tramadol, oxycodone or fentanyl;
ccc) Kappa opioid agonists, such as nalfurafine, nalbuphine or ketazocine;
ddd) Neurokinin receptor 1 antagonists, such as aprepitant or fosaprepitant; and
eee) Dihydropteroate synthase inhibitors, such as dapsone.
a) The other therapeutic agent is preferably chosen from Dihydrofolate reductase inhibitors, such as Methotrexate or CH-1504;
b) Immunosuppressants, such as Imuran (azathioprine), cyclophosphamide, sirolimus or Purinethol (6-mercaptopurine or 6-MP);
c) Corticoids and glucocorticoids such as prednisone, methylprednisolone, fluticasone, dexamethasone, mometasone, budesonide, ciclesonide or beta-metasone;
d) Anti-tumor necrosis factor-alpha (Anti-TNF-alpha) monoclonal antibodies such as Infliximab, Adalimumab or Certolizumab pegol;
e) Soluble Tumor necrosis factor-alpha (TNF-alpha) Antagonists such as Etanercept;
f) Anti-CD20 (lymphocyte protein) monoclonal antibodies such as Rituximab, Ocrelizumab, Ofatumumab or TRU-015
g) Anti-BAFF/BlyS such as belimumab, tabalumab, or blisibimod
h) Anti-TACl such as atacicept
i) Anti-BAFF receptor such as VAY736
j) Anti-CD19 such as MEDI-551
k) Anti-ICOSL such as AMG-557
l) Anti-FasL monoclonal antibodies
m) Btk inhibitors like ibrutinib
n) Calcineurin inhibitors such as cyclosporine A, pimecrolimus or tacrolimus;
o) Inosine-monophosphate dehydrogenase (IMPDH) inhibitors, such as mycophenolate mophetyl, ribavirin, mizoribine or mycophenolic acid;
p) Tetracyclines, such as methacycline, doxycycline or minocycline; and
q) Dihydropteroate synthase inhibitors, such as dapsone;

The therapeutic agent is more preferably chosen from prednisolone, betamethasone, dexamethasone, methylprednisolone, azathioprine, mizoribine, mycophenolate mofetil, mycophenolic acid, dapsone, acitretin, cyclophosphamide, immunoglobulin (Ig), thalidomide, tetracycline and rituximab.

The most preferable therapeutic agents for combination are prednisolone and azathioprine.

The invention also encompasses a pharmaceutical composition for use in the treatment of an immunobullous skin disease mediated by autoantibodies as defined herein by oral administration, which composition comprises a compound as defined herein and a pharmaceutically acceptable carrier as defined herein.

In an embodiment of the present invention the pharmaceutical composition further comprises a therapeutically effective amount of one or more other therapeutic agents, as defined above.

The invention is also directed to combinations for use in the treatment of an immunobullous skin disease mediated by autoantibodies as defined herein by oral administration, the combinations comprising a compound as defined herein and a therapeutically effective amount of one or more other therapeutic agents as defined above. The invention is also directed to pharmaceutical compositions for use in the treatment of an immunobullous skin disease mediated by autoantibodies as defined herein by oral administration, the compositions comprising such combinations.

The present invention is also directed to use of a compound, composition or combination as defined herein, for the manufacture of a medicament for the treatment of an immunobullous skin disease mediated by autoantibodies as defined herein by oral administration.

The invention also encompasses a method of treating an immunobullous skin disease mediated by autoantibodies as defined herein, which method comprises administering orally to a patient in need thereof an effective amount of a compound, composition or combination as defined herein.

The invention also provides a method of treating an immunobullous skin disease mediated by autoantibodies as defined herein, which method comprises:

(a) selecting a patient suffering from or susceptible to an immunobullous skin disease mediated by autoantibodies, and
(b) administering orally to a patient in need thereof an effective amount of a compound, composition or combination as defined herein.

The compound for use in the method of the present invention, and the other optional therapeutic agents may be administered together in the same pharmaceutical composition or in different compositions intended for separate, simultaneous, concomitant or sequential administration by the same or a different route.

One execution of the present invention consists of a kit of parts comprising a compound as defined herein together with instructions for simultaneous, concurrent, separate or sequential use in combination with another therapeutic agent useful in the treatment of an immunobullous skin disease mediated by autoantibodies as defined herein.

Another execution of the present invention consists of a package comprising a compound as defined herein and another therapeutic agent useful in the treatment of an immunobullous skin disease mediated by autoantibodies as defined herein.

The pharmaceutical formulations may conveniently be presented in unit dosage form and may be prepared by any of the methods well known in the art of pharmacy.

Pharmaceutical compositions suitable for the delivery of compounds of the invention and methods for their preparation will be readily apparent to those skilled in the art. Such compositions and methods for their preparation can be found, for example, in Remington: The Science and Practice of Pharmacy, 21st Edition, Lippincott Williams & Wilkins, Philadelphia, Pa., 2001.

The pharmaceutically acceptable excipients which are admixed with the active compound or salts of such compound, to form the compositions of this invention are well-known per se and the actual excipients used depend inter alia on the intended method of administering the compositions. Examples, without limitation, of excipients include calcium carbonate, calcium phosphate, various sugars and types of starch, cellulose derivatives, gelatin, vegetable oils and polyethylene glycols.

Additional suitable carriers for formulations of the compounds of the present invention can be found in Remington: The Science and Practice of Pharmacy, 21st Edition, Lippincott Williams & Wilkins, Philadelphia, Pa., 2001.

The compounds for use in the method of the present invention (and the compositions and combinations) are administered orally, as syrups, tablets, capsules, lozenges, controlled-release preparations, fast-dissolving preparations, etc.

Formulations of the present invention which are suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound in a liquid carrier, for example ethanol, peanut oil, olive oil, glycerine or water with flavouring or colouring agent.

Where the composition is in the form of a tablet, any pharmaceutical carrier routinely used for preparing solid formulations may be used. Examples of such carriers include acacia, lactose, D-glucose (dextrose), sucrose, fructose, galactose, gelatine, starch, calcium carbonate, dibasic calcium phosphate, calcium sulphate, magnesium stearate, magnesium carbonate, isomalt, mannitol, maltitol, stearic acid, sorbitol, talc, xylitol, and mixtures thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent.

Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the composition is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, incorporated in a soft gelatine capsule.

Preferably the composition is in unit dosage form, for example a tablet or capsule, so that the patient may administer a single dose.

The amount of each active which is required to achieve a therapeutic effect will, of course, vary with the particular active, the route of administration, the subject under treatment, and the particular disorder or disease being treated.

Effective doses are normally in the range of 0.01-2000 mg of active ingredient per day. Daily dosage may be administered in one or more treatments, preferably from 1 to 4 treatments, per day. Preferably, the active ingredients are administered once or twice a day, more preferably once a day.

When combinations of actives are used, it is contemplated that all active agents would be administered at the same time, or very close in time. Alternatively, one or two actives could be taken in the morning and the other (s) later in the day. Or in another scenario, one or two actives could be taken twice daily and the other (s) once daily, either at the same time as one of the twice-a-day dosing occurred, or separately. Preferably at least two, and more preferably all, of the actives would be taken together at the same time. Preferably, at least two, and more preferably all actives would be administered as an admixture.

The following preparations forms cited as composition (formulation) examples are given in order to provide a person skilled in the art with a sufficiently clear and complete explanation of the present invention, but should not be considered as limiting of the essential aspects of its subject, as set out in the preceding portions of this description.

It is a surprising finding of the present invention that the compounds of the invention have significantly improved efficacy against immunobullous skin diseases mediated by autoantibodies when administered orally. The oral efficacy of the compounds of the invention is higher than the efficacy of the compounds when delivered by topical administration.

### Composition Example 1

50,000 capsules, each containing 100 mg LAS191954, methanesulfonate (active ingredient), were prepared according to the following formulation:

| | | |
|---|---|---|
| Active ingredient | 5 | Kg |
| Lactose monohydrate | 10 | Kg |
| Colloidal silicon dioxide | 0.1 | Kg |
| Corn starch | 1 | Kg |
| Magnesium stearate | 0.2 | Kg |

### Procedure

The above ingredients were sieved through a 60 mesh sieve, and were loaded into a suitable mixer and filled into 50,000 gelatine capsules.

### Composition Example 2

50,000 tablets, each containing 50 mg of LAS191954, methanesulfonate (active ingredient), were prepared from the following formulation:

| | | |
|---|---|---|
| Active ingredient | 2.5 | Kg |
| Microcrystalline cellulose | 1.95 | Kg |
| Spray dried lactose | 9.95 | Kg |
| Carboxymethyl starch | 0.4 | Kg |
| Sodium stearyl fumarate | 0.1 | Kg |
| Colloidal silicon dioxide | 0.1 | Kg |

### Procedure

All the powders were passed through a screen with an aperture of 0.6 mm, then mixed in a suitable mixer for 20 minutes and compressed into 300 mg tablets using a 9 mm disc and flat bevelled punches. The disintegration time of the tablets was about 3 minutes.

Modifications, which do not affect, alter, change or modify the essential aspects of the compounds, combinations or pharmaceutical compositions described, are included within the scope of the present invention.

The following examples illustrate the invention

### Example 1—In Vitro Pharmacology Studies

The pharmacology of LAS191954 has been investigated in a range of in vitro studies.

### PI3Kδ Enzyme Residence Time

LAS191954 showed a residence time (time interval in which dissociation of 50% of the inhibitor occurs) in p1106 of 12 min or 17 min, whereas residence time was <1.4 min for the other three class I isoforms.

### Enzymatic and Cellular Potencies

Enzymatic potency on the four Class I PI3K recombinant human isoforms was determined by homogenous time-resolved fluorescence with a compound pre-incubation time of 30 min (Table 1). LAS191954 showed a potency on the target of 2.6 nM, with the highest selectivity versus PI3K p110α and the lowest versus PI3K p110γ and p110β, similarly.

TABLE 1
Enzymatic potencies of LAS191954 in the four PI3K isoforms

| Enzyme | IC₅₀ (nM) | Selectivity vs PI3Kδ (fold) |
|---|---|---|
| PI3K p110δ | 2.57 | 1 |
| PI3K p110α | 8220 | 3198 |
| PI3K p110β | 94.2 | 37 |
| PI3K p110γ | 71.7 | 28 |

Cellular potencies were determined in established cellular assays (Table 2). A primary PI3Kδ-dependent cellular assay was set up based on M-CSF-induced AKT phosphorylation, a downstream effector of PI3Kδ, in THP-1 cells. An IC₅₀ of 7.8 nM was obtained indicating that the compound was highly permeable. To evaluate the cellular inhibition of PI3Kβ, an assay based on stimulation of HUVEC cells with sphingosine-1-P was employed. The results indicated that the cellular selectivity for the β isoform was 38-fold.

The main receptor on the surface of B cells is the BCR composed of a membrane immunoglobulin (Ig) and an Igα/Igβ heterodimer. The BCR is responsible for antigen recognition and binding. Signaling pathways associated with the BCR are crucial for B cell development, activation, proliferation, differentiation (e.g., memory and plasma B cells) and apoptosis. In naïve B cells, ligation of the BCR by cognate antigens initiates a series of responses/signal cascades that will induce cells to proliferate and differentiate, and will ultimately lead to the production of antibodies specific for the antigen. The PI3Kδ kinase is involved in the activation of B cells upon antigen binding to the BCR and thus inhibitors of PI3Kδ are expected to inhibit BCR activation in vitro.

The effect of LAS191954 on the function of human B cells was assessed in vitro by crosslinking the B-cell receptor with either anti-IgM or anti-IgD antibodies and assessing the early activation marker CD69 in the CD19+ B cell subset by flow cytometry. In isolated PBMC, the compound showed an IC₅₀ of 4.6 nM. Similar assays performed in a human whole blood context showed IC₅₀ of 47 nM for IgD and 34 nM for IgM. Plasma protein binding is the major factor accounting for the difference in potency between isolated PBMC and whole blood assays. These data indicate that LAS191954 is active in PBMCs in whole blood to inhibit B cell activation and antibody production.

In a functional assay on human neutrophils assessing the immune complex-induced ROS (reactive oxygen species) release, LAS191954 showed a potency of 11 nM, suggesting that PI3Kδ might be the only isoform involved in this effect.

TABLE 2
Cellular potencies of LAS191954

| | | | Isoform | IC₅₀ | Nbr |
|---|---|---|---|---|---|
| Cell type | Stimulus | Read out | involved | (nM) | tests |
| THP-1 | M-CSF | Phosphorylated | PI3Kδ | 7.8 | 2 |
| | | AKT | | | |
| HUVEC | S1P | Phosphorylated | PI3Kβ | 295 | 3 |
| | | AKT | | | |
| CD19+ cells | Anti-IgD | CD69 surface | PI3Kδ | 4.6 | 2 |
| (PBMC) | | expression | | | |
| CD19+ cells | Anti-IgD | B cell CD69 | PI3Kδ | 47 | 3 |
| (Human | | surface | | | |
| whole blood) | | expression | | | |
| CD19+ cells | Anti-IgM | B cell CD69 | PI3Kδ | 34 | 8 |
| (Human | | surface | | | |
| whole blood) | | expression | | | |
| Human | Immune | ROS release | PI3Kδ | 11 | 6 |
| neutrophils | complexes | | | | |

### General Selectivity

Activity of LAS191954 was assessed at a single concentration of 10 μM in:
81 GPCR receptors, 8 ion channels and 5 transporters (Cerep)
S273 protein and lipid kinases (Millipore, Invitrogen and ProQinase)

### Cytotoxicity

In an assay assessing cytotoxicity of CHO cells after 24 h compound incubation, LAS191954 caused negligible cytotoxicity at all concentrations tested causing a maximum of 27% cell death at the highest tested concentration of 100 μM. This result indicates that the compound is not expected to be cytotoxic at estimated therapeutic plasma/tissue concentrations attained. No dose-response is observed across concentrations.

### Example 2—In Vivo Pharmacology Studies

The pharmacology of LAS191954 has been investigated in vivo using a range of studies listed in Table 3 below. The results of these studies are summarized in Table 4.

TABLE 3
In Vivo Studies of LAS191954

Effect Of Oral Administration Of LAS191954 on Concanavalin A
Induced Plasma Interleukin-2 (IL-2) Release In Wistar Rats
Evaluation of the Effect of LAS191954 On Primary and Secondary
T cell Dependent Antibody Response (TDAR) in Mice
Effect Of LAS191954 on A Murine Model Of Spontaneous
autoimmune disease

TABLE 4
Summary of In Vivo Studies Performed and
Inhibitory Doses Reported for LAS191954

| | | Type of | ID₅₀ |
|---|---|---|---|
| Study | Administration | model | (mg/kg) |
| Concanavalin A-induced IL2 | Single | Mechanistic | 0.13 |
| production in rats | | | |
| Primary IgM antibody | Repeated | Immune | 0.12 |
| responses to KLH | | response | |
| Primary IgG antibody | Repeated | Immune | 0.17 |
| responses to KLH | | response | |
| Secondary IgG antibody | Repeated | Immune | <0.3 |
| responses to KLH | | response | |
| Anti-Dsg3 and anti-dsDNA | Repeated | Autoimmune | <3 |
| autoantibody production in | | response | |
| Mrl/Lpr model | | | |

### LAS191954 Inhibits T-Cell Dependent Antibody Responses in Mice

The TDAR (T-Dependent Antibody Response) assay in mice using the KLH as antigen was selected to further explore the effect of LAS191954 on the function of the immune system. This assay allows a global assessment of the effect of a drug candidate on antigen presentation, helper T lymphocyte function and B lymphocyte dependent antibody production.

According to the kinetics of the specific antibody responses, the effect on primary specific IgM anti-KLH was analyzed on day+5 post immunization (PI) after 4 days of daily treatment with LAS191954 (0.03-10 mg/kg), and the effect on primary specific IgG was assessed on day+15 PI after 14-day dosing period (0.03-1 mg/kg). In both cases, the administration of the test compound started on the day of sensitization (day +1, KLH 2 mg/mouse, intravenously). LAS191954 induced a significant dose-dependent decrease in the primary IgM (ID₅₀=0.12 mg/kg) and IgG (ID₅₀=0.17 mg/kg) responses to KLH without apparent effects on the general health status of the animals. The decrease in the primary IgM anti-KLH response was accompanied by lower WBC counts mainly due to reduced number of peripheral blood lymphocytes. In contrast, no apparent effect on lymphocyte count was observed after treatment with LAS191954 in the study where specific IgG were analyzed. A possible reason for this discrepancy is that the lymphocyte count of the concurrent vehicle group of the latter study was abnormally lower than usual, which could mask a potential effect of the test compound on this parameter.

The effect of LAS191954 was subsequently assessed on the secondary TDAR assay in mice. This assay included two immunizations with KLH separated 15 days apart (50 μg KLH/animal, intraperitoneally) and specific IgG anti-KLH levels were measured on day +11 after the second immunization. Administration of test compound (0.3 and 3 mg/kg) started on the day of second immunization (day +1) and then once daily for the next 9 days. LAS191954 induced a significant decrease in secondary specific IgG anti-KLH response accompanied by reduced lymphocyte counts with an ID₅₀<0.3 mg/kg.

In the same TDAR assay protocols, a representative corticosteroid did not induce significant changes in the anti-KLH antibody response, while decreasing peripheral lymphocyte count and thymus weight.

### Example 3—Inhibition of Specific Dsq3 Autoantibody Production in a Spontaneous Autoimmune Disease Model

The MRL/lpr mouse model was selected as a model of efficacy to demonstrate amelioration of autoimmune-related features, in particular, production of autoantibodies. The primary endpoint of this study was assessment of autoantibody production, including pemphigus-specific anti-Dsg3 antibodies.

Mice were randomized to receive vehicle alone, 3 mg/kg LAS191954, or 10 mg/kg prednisolone orally once a day for 6 weeks. The dose of LAS191954 was selected to ensure complete PI3Kδ coverage for 24 h when administered once a day. The prednisolone dose was selected based on previous reports and corresponds to a high CS dose in humans.

As autoantibodies develop progressively and may follow a different course in each animal, anti-dsDNA antibody levels were measured on week 12 and used to uniformly distribute animals to dosing groups. At week 13, daily treatments were initiated and continued for 6 weeks. Antibodies to dsDNA and Dsg3 were measured at weeks 12, 15, 17 and 19. Skin lesions were inspected visually throughout the study. Effects on other parameters such as proteinuria, as well as general hematological, serological, and histological signs were evaluated at study completion.

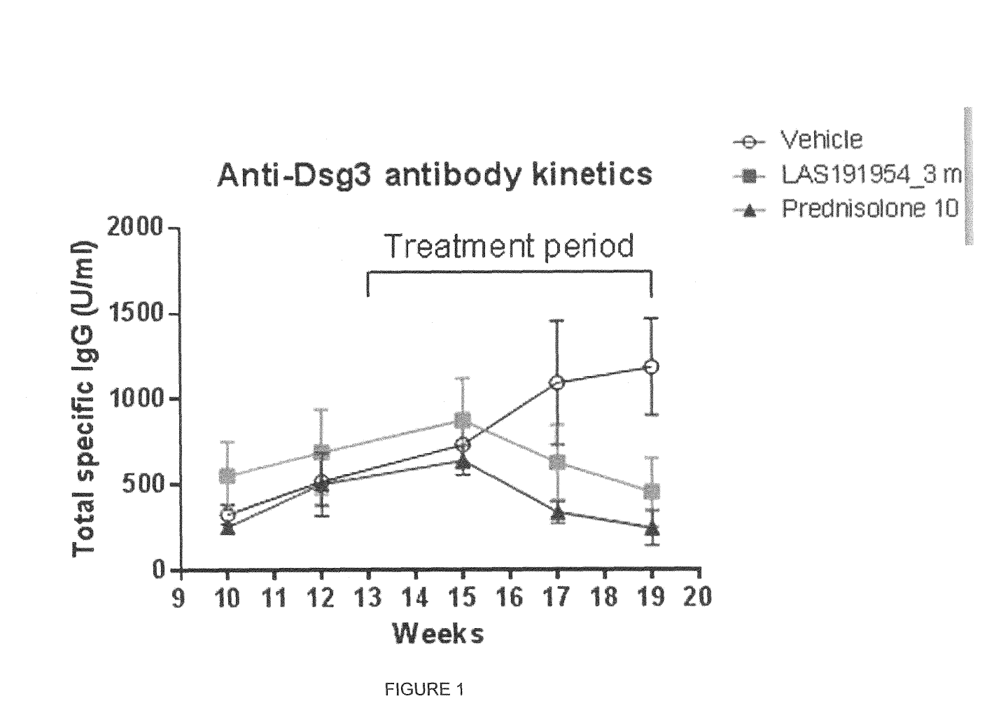

Kinetic analysis of autoantibody production demonstrated that anti-dsDNA antibody levels were approximately 2,000-fold higher than anti-Dsg3 antibodies and increased steadily between weeks 12 and 19. Daily administration of LAS191954 for 6 weeks at a dose of 3 mg/kg significantly reduced anti-dsDNA and anti-Dsg3 antibody production (see FIGS. 1&2). When the Area Under Curve encompassing weeks 12 to 19 was calculated and normalized for initial week 12 antibody titer for each individual, LAS191954 led to a 47.5% and a 66% inhibition of anti-Dsg3 and anti-dsDNA antibodies, respectively, similar to prednisolone (49.5 and 47%, respectively) (Table 5).

TABLE 5
Inhibition (%) of Autoantibody Production
(Normalized AUCw 12-19) versus Vehicle

| | LAS191954 3 mg/kg | Prednisolone 10 mg/kg |
|---|---|---|
| Anti Dsg3 total IgG | 47.5 ± 10.4% * | 49.5 ± 10% * |
| Anti dsDNA total IgG | 65.6 ± 3.1% *** | 46.9 ± 10.9% ** |

*** p < 0.001;
** p < 0.01;
* p < 0.05 using one-way ANOVA and Dunnet post-test versus vehicle group

When absolute specific IgG levels were measured, LAS191954 reduced the average levels of anti-dsDNA and anti-Dsg3 specific IgGs on the last week of administration below those at the start of treatment (Table 6).

TABLE 6
Absolute Specific IgG Levels at the Beginning and End of Treatment
Absolute IgG levels (U/ml) (Mean ± SEM)

| | Anti-dsDNA | | Anti-Dsg3 | |
|---|---|---|---|---|
| | Week 12 | Week 19 | Week 12 | Week 19 |
| Vehicle | 369.253 ± 52.919 | 2,750.673 ± 745.899 | 515 ± 140 | 1185 ± 284 |
| LAS191954 | 370.152 ± 52.401 | 333.806 ± 106.959 | 687 ± 252 | 450 ± 201 |
| 3 mg/kg | | | | |
| Prednisolone | 372.796 ± 56.638 | 933.755 ± 343.424 | 501 ± 188 | 242 ± 101 |
| 10 mg/kg | | | | |

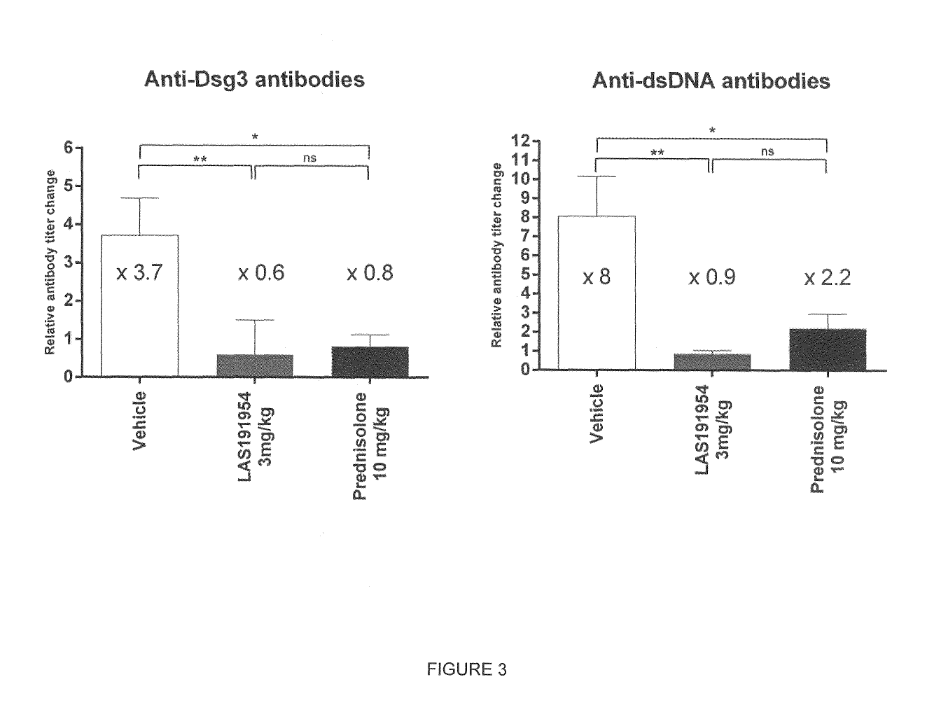

FIG. 3 shows the fold change in antibody titers at week 19 versus titers at the initiation of treatment. Whereas the anti-Dsg3 antibody titers increased approximately four-fold in the vehicle treated animals, LAS191954 and prednisolone induced a mean decrease of 40% and 20%, respectively, in antibody levels below those at the beginning of treatment. Likewise, anti-dsDNA antibody titers increased about 8-fold, whereas LAS191954 caused a 10% reduction and prednisolone doubled the levels at the end of treatment. For each individual, the ratio between antibody titer at Week 19 and that at Week 12 was calculated. (Values represent mean of ratios for each treatment group ±SEM. * p<0.05; **p<0.01; ns nonstatistically significant.)

This demonstrates that prolonged daily treatment with LAS191954 is able to significantly reduce anti-Dsg3 autoantibody production in a spontaneous model of autoimmune disease that does not rely on active immunization. Antibodies to both dsDNA and most importantly Dsg3, the specific antigen in PV, were reduced with similar efficiency.

### Example 4—Immunization-Induced Mouse Model of Epidermolysis Bullosa Acquista (EBA)

LAS191954 was tested in an immunization-induced mouse model of epidermolysis bullosa acquista (EBA) in B6.SJL-H2s mice to demonstrate the link between PI3Kδ inhibition and amelioration of autoantibody-mediated cutaneous lesions.

### Materials and Methods

### Animal Experiments

Induction of experimental EBA was performed as described in Iwata H, Bieber K, Tiburzy B et al., J Immunol. 2013; 191:2978-2988. Briefly, 6-10 week B6.SJL-H2s mice were immunized with an emulsion of a recombinant protein encompassing the vWFA2 binding domain of mouse type VII collagen (COL7) in adjuvant (Titermax). After immunization, mice were weekly evaluated for the presence and extend of clinical disease, measured as percentage of body surface affected by skin lesions (erythema, blisters, erosions and crusts). When 2% or more of the body surface area was affected by skin lesions, the mouse was randomly allocated to one of the treatment groups:
Vehicle to serve as untreated control (n=5)
Methylprednisolone (MP, orally at 20 mg/kg/day) to serve as reference treatment (n=6)
LAS191954 orally at 3 mg/kg/day (n=6)

Treatments were carried out over a 6-week period, and mice were evaluated for the extend of clinical disease (primary endpoint) weekly. Clinical manifestations were scored 0 to 5, corresponding to 0%, <1%, ≧1% to <5%, ≧5% to <10%, ≧10% to <20% of body surface area affected, respectively. Area under Curve (AUC) was calculated from the score at inclusion, 1, 2, 3, 4, 5 and 6 weeks after allocation to treatment. For better comparability between experiments, the affected body surface area at weeks 1-6 was related to that at inclusion (set at 1).

Body weight was monitored weekly during treatment.

### Results

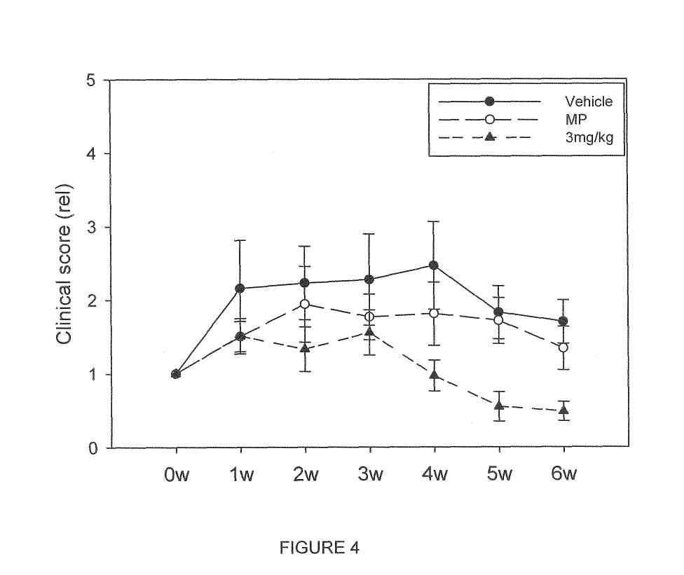

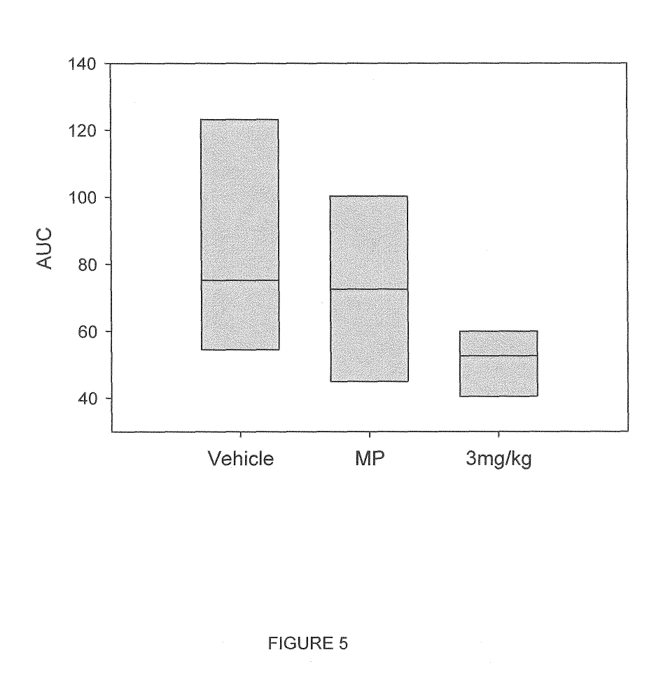

In vehicle-treated mice, the relative clinical score increased from 1 to 1.7 at the end of the 6 week treatment period with a maximum index of 2.5 observed at 4 weeks of treatment (FIGS. 4 and 5).

FIG. 4 shows the percentage of body surface area affected by skin lesions in relation to the score at inclusion to treatment. Disease severity increases in vehicle-treated group during the 6 week treatment period. Methylprednisolone modestly reduced clinical severity during the 6 week treatment period versus the vehicle-treated group, although it was not statistically significant. In contrast, LAS191954 progressively and significantly (p<0.001 for weeks 4, 5 and 6) reduced the clinical severity over the same period, obtaining a final score below the initial one, i.e. even beyond the initial clinical score (mean±SEM), indicating a clear trend towards normalization.

FIG. 5 shows the overall disease activity, expressed as AUC derived from graphs in FIG. 4. (Median ±quartiles). In accordance with the time-course results, Area Under Curve calculation showed a significant reduction in the accumulative clinical score over time with LAS191954 treatment versus vehicle.

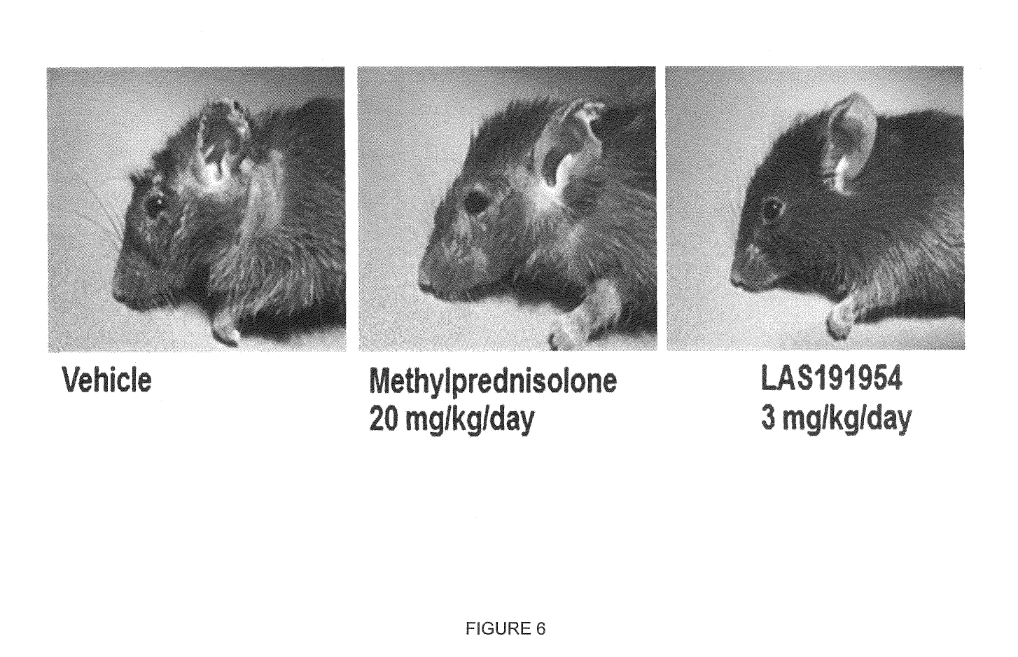

FIG. 6 shows representative clinical manifestations of the three treatment groups at the end of the treatment period.

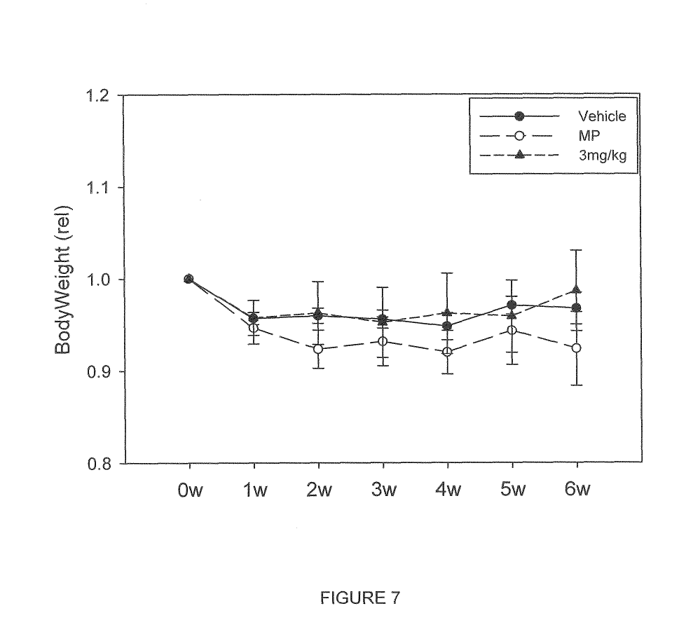

Body weight gain was not altered by LAS191954 administration over time. In contrast, methylprednisolone diminished the body weight gain especially at the beginning of treatment (FIG. 7). The LAS191954-treated group showed a similar behavior to the vehicle-treated group with modest gain weights along the treatment period. The methylprednisolone-treated group showed lower gain weight than the vehicle group, especially during the first two weeks of treatment.

### Conclusion

LAS191954 ameliorates the cutaneous disease manifestations in an induced model of epidermolysis bullosa acquisita, an autoantibody-mediated bullous disease model. The effect is better than that induced by treatment with a high dose corticosteroid and shows a clear trend towards time-dependent clinical normalization. Taken together, these results provide a direct link between PI3Kδ inhibition and clinical efficacy in a cutaneous bullous disease.

### Example 5—Neonatal Passive Transfer Model

LAS191954 showed no direct effect on anti-Dsg3 antibody-induced skin damage.

TABLE 7

| | All experiments (n = 4 independent litters) | | | |
|---|---|---|---|---|
| | With | Total mice | % with | |
| | blisters | treated | blisters | P = |
| Normal Human (NH) IgG | 0 | 5 | 0% | — |
| PV IgG | 5 | 7 | 71% | 0.04¹ |
| PV IgG + LAS191954 | 6 | 8 | 75% | ns² |
| (1 mg/kg) | | | | |
| PV IgG + LAS191954 | 8 | 8 | 100% | ns² |
| (10 mg/kg) | | | | |

Statistical significance was calculated with 2-Way ANOVA using Holm-Sidak post-test analysis (vs ¹NH IgG, ²PV IgG; ns: non significant)

1.-15. (canceled)

16. A method for treating a subject afflicted with immunobullous skin disease mediated by autoantibodies comprising orally administering to the subject a therapeutically effective amount of a compound chosen from an inhibitor of phosphoinositide 3-kinase delta or a pharmaceutically acceptable salt and/or solvate thereof.

17. The method according to claim 16, wherein the immunobullous skin disease is mediated by anti-Dsg autoantibodies.

18. The method according to claim 16, wherein the immunobullous skin disease mediated by autoantibodies is chosen from pemphigus vulgaris, pemphigus vegetans, pemphigus foliaceus, endemic pemphigus foliaceus, intercellular IgA dermatosis, paraneoplastic pemphigus, bullous pemphigoid, mucous membrane pemphigoid, pemphigoid gestationis, linear IgA disease, epidermolysis bullosa acquisita, bullous systemic lupus erythematosus and dermatitis herpetiformis.

19. The method according to claim 16, wherein the immunobullous skin disease mediated by autoantibodies is pemphigus vulgaris.

20. The method according to claim 16, wherein the compound is chosen from: LAS191954, idelalisib, duvelisib, enzastaurin, rigosertib, buparlisib, taselisib, dactolisib, copanlisib, pictrelisib, apitolisib, sonolisib, voxtalisib, ZSTK-474, GSK-2269557, UCB-5857, RV-1729, RP-6530, omipalisib, SB-2343, WX-037, CAL-120, PWT-33597, CUDC-907, AMG-319, puquitinib, pilaralisib, RP-5264, GDC-0084 (or GDC-7666), LY-3023414, PQR-309, DS-7423, XL-499, KAR-4141, RP-5090, PWT-143, IPI-443, RP-6503, ONO-146040, SPR-965, LOR-220, SF-2626, X-339, X-480, PQR-401, INCB-050465, LS-008, CLR-457, PCN-5603, 7-hydroxystaurosporine, PF-04691502, TG-100115, BGT-226, SF-1126, PKI-179, panulisib, or a pharmaceutically acceptable salt and/or solvate thereof.

21. The method according to claim 16, wherein the compound is chosen from LAS191954 or a pharmaceutically acceptable salt and/or solvate thereof.

22. The method according to claim 16, wherein the compound is a pharmaceutically acceptable crystalline addition salt of LAS191954 with a sulfonic acid derivative chosen from methanesulfonic acid, naphthalene-2-sulfonic acid and para-toluenesulfonic acid, or a pharmaceutically acceptable solvate thereof.

23. The method according to claim 16, wherein the compound is chosen from LAS191954 methanesulfonate, or a pharmaceutically acceptable solvate thereof.

24. The method according to claim 16, wherein the compound is chosen from LAS191954 naphthalene-2-sulfonate, or a pharmaceutically acceptable solvate thereof.

25. The method according to claim 16, wherein the compound is chosen from LAS191954 para-toluenesulfonate, or a pharmaceutically acceptable solvate thereof.

26. The method according to claim 16, wherein the compound is chosen from LAS191954 or a pharmaceutically acceptable salt and/or solvate thereof and the immunobullous skin disease mediated by autoantibodies is pemphigus vulgaris.

27. The method according to claim 16, wherein the compound is co-administered with a therapeutically effective amount of at least one additional therapeutic agent chosen from:
a) Dihydrofolate reductase inhibitors,
b) Immunosuppressants,
c) Corticoids and glucocorticoids,
d) Anti-tumor necrosis factor-alpha (Anti-TNF-alpha) monoclonal antibodies,
e) Soluble Tumor necrosis factor-alpha (TNF-alpha) Antagonists,
f) Anti-CD20 (lymphocyte protein) monoclonal antibodies,
g) Anti-BAFF/BlyS,
h) Anti-TACl,
i) Anti-BAFF receptor,
j) Anti-CD19,
k) Anti-ICOSL,
l) Anti-FasL monoclonal antibodies,
m) Btk inhibitors,
n) Calcineurin inhibitors,
o) Inosine-monophosphate dehydrogenase (IMPDH) inhibitors,
p) Tetracyclines, and
q) Dihydropteroate synthase inhibitors.

28. The method according to claim 27, wherein the dihydrofolate reductase inhibitor is chosen from Methotrexate or CH-1504.

29. The method according to claim 27, wherein the immunosuppressant is chosen from Imuran (azathioprine), cyclophosphamide, sirolimus or Purinethol (6-mercaptopurine or 6-MP).

30. The method according to claim 27, wherein the corticoid and glucocorticoid is chosen from prednisolone, prednisone, methylprednisolone, fluticasone, dexamethasone, mometasone, budesonide, ciclesonide or beta-metasone.

31. The method according to claim 27, wherein the anti-tumor necrosis factor-alpha (Anti-TNF-alpha) monoclonal antibody is chosen from Infliximab, Adalimumab or Certolizumab pegol.

32. The method according to claim 27, wherein the soluble tumor necrosis factor-alpha (TNF-alpha) antagonist is Etanercept.

33. The method according to claim 27, wherein the anti-CD20 (lymphocyte protein) monoclonal antibody is chosen from Rituximab, Ocrelizumab, Ofatumumab or TRU-015.

34. The method according to claim 27, wherein the anti-BAFF/BlyS is chosen from belimumab, tabalumab, or blisibimod.

35. The method according to claim 27, wherein the anti-TACl is atacicept.

36. The method according to claim 27, wherein the anti-BAFF receptor is VAY736.

37. The method according to claim 27, wherein the anti-CD19 is MEDI-551.

38. The method according to claim 27, wherein the anti-ICOSL is AMG-557.

39. The method according to claim 27, wherein the Btk inhibitor is ibrutinib.

40. The method according to claim 27, wherein the calcineurin inhibitor is chosen from cyclosporine A, pimecrolimus or tacrolimus.

41. The method according to claim 27, wherein the inosine-monophosphate dehydrogenase (IMPDH) inhibitor is chosen from mycophenolate mophetyl, ribavirin, mizoribine or mycophenolic acid.

42. The method according to claim 27, wherein the tetracycline is chosen from methacycline, doxycycline or minocycline.

43. The method according to claim 27, wherein the dihydropteroate synthase inhibitor is dapsone.
